(19) United States
(12) Patent Application Publication
(10) Pub. No.: US 2025/0032483 A1
(43) Pub. Date: Jan. 30, 2025

(54) COMPOSITIONS AND METHODS FOR TREATING PAIN

(71) Applicant: ICAHN SCHOOL OF MEDICINE AT MOUNT SINAI, New York, NY (US)

(72) Inventors: Randal Alexander SERAFINI, New York, NY (US); Venetia ZACHARIOU, New York, NY (US); Justin FRERE, New York, NY (US); Benjamin TENOEVER, New York, NY (US)

(73) Assignee: ICAHN SCHOOL OF MEDICINE AT MOUNT SINAI, New York, NY (US)

(21) Appl. No.: 18/715,236

(22) PCT Filed: Dec. 2, 2022
(86) PCT No.: PCT/US22/51701

§ 371 Date: May 31, 2024

Related U.S. Application Data
(60) Provisional application No. 63/285,790, filed on Dec. 3, 2021.

(51) Int. Cl. A61K 31/497; A61P 25/04

(52) U.S. Cl. CPC A61K 31/497; A61P 25/04

(57) ABSTRACT

Disclosed herein are methods and compositions for preventing pain induction, interrupting pain signaling stemming from various insults, or alleviating pain perception in a subject.

### RELATED APPLICATION

This application is a § 371 national stage of PCT International Application No. PCT/US22/051701, filed Dec. 2, 2022, which claims priority to U.S. Provisional Application No. 63/285,790, filed Dec. 3, 2021, which are incorporated herein by reference in their entirety.

### BACKGROUND

Current estimates suggest that 1 in 5 adults suffer from chronic pain globally, with 1 in 10 adults receiving a new diagnosis every year. Despite this, pain treatments remain limited and relatively ineffective or potentially harmful in the long-term. For example, non-steroidal anti-inflammatory drugs (NSAIDs) are effective in short bursts for selective pain indications and can cause significant side effects when used long-term. And while opioids have a longer window of activity and higher efficacy, they also trigger severe side effects, including analgesic tolerance, addiction, and overdose. Few of the existing compounds used for pain relief have an immediate onset of action and can be safely used for long periods of treatment.

Sever acute respiratory syndrome coronavirus 2 (SARS-COV-2) infection has been associated with sensory abnormalities that manifest in both acute and long-lasting phenotypes. Recent research has shown that a significant number of actively infected patients suffering from both mild and severe infections experience sensory-related symptoms such as headache, visceral pain, Guillain-Barre syndrome (GBS), nerve pain, and polyneuritis. While these symptoms subside after clearance of infection in most patients, they have been noted to arise in or persist to sub-acute or chronic timepoints for many. Sensory-related symptomology is thus a major component of long coronavirus disease (COVID). However, the mechanisms by which coronaviruses, and specifically SARS-COV-2, induce abnormal sensation are poorly understood. Recent data suggest that virus replication in the airways results in the dissemination of viral ribonucleic acid (RNA) and the induction of an antiviral transcriptional response in distal tissues, including the brain, which may underly central nervous system (CNS)-related pathologies, such as demyelinating lesions and brain hemopathologies, observed among COVID-19 patients.

Despite the large number of studies investigating CNS infiltration during SARS-CoV-2 infection, little clinical or pre-clinical literature has investigated penetration capabilities of SARS COV-2 into the peripheral and central nervous systems, particularly sensory components such as the dorsal root ganglia (DRGs) and spinal cord (SC). SARS-COV-2 has been shown to infect human neuronal cells in vitro and to induce a robust in vivo systemic inflammatory response. The present disclosure provides insights into the sensory-altering mechanisms induced by respiratory SARS-COV-2 infection and thus reveals new approaches and compounds for addressing pain. In particular, the description below demonstrates that inhibition of Interleukin Enhancer Binding Factor 3 (ILF3), a potent regulator of anti-viral responses and neuronal activity, expression or activity is an effective modality for treating or preventing sensory manifestations of pain in pre-clinical models of peripheral inflammation, interferon-induced myalgia, and surgical incisions.

### SUMMARY

The present disclosure solves the above-mentioned issues related to the treatment or prevention of emergent and chronic pain by providing novel pain treatment methods that operate via the inhibition of ILF3 expression or activity using various mechanisms. The present disclosure provides novel and highly effective methods for providing immediate and sustained pain alleviation in a variety of indications, including pain symptomatology due to peripheral inflammation, neuropathy, and viral infection.

In an aspect, the present disclosure provides a method for preventing pain induction, interrupting pain signaling, or alleviating pain perception in a subject by inhibiting the expression, activity, or function of ILF3. In some embodiments, the type of pain is selected from the group consisting of: neuropathic pain, due to factors including but not limited to chemotherapy, nerve injury, nerve compression, autoimmune responses, retroviral infection: postoperative pain: myalgia: chronic pain: emergent pain: systemic pain; and pain associated with inflammation, infection, post-viral conditions (including, for example, myalgic encephalitis/chronic fatigue syndrome (ME/CFS) and post-acute sequelae of COVID-19 (PASC)), or disease. The preventing, interrupting, or alleviating comprises administering to the subject a polynucleotide, polypeptide, chemical compound, nanoparticles, delayed-release compound, or pharmaceutical.

In some embodiments, the preventing, interrupting, or alleviating comprises administering a compound selected from the group consisting essentially of: YM155, genistein, ivermectin, piroxicam, resveratrol, tamibarotene, thapsigargin, thimerosal, valproic acid, corticosteroids (such as prednisone, hydrocortisone, cortisone, methylprednisolone, dexamethasone, prednisolone), JAK/TYK inhibitors (such as baricitinib, tofacitinib, upadacitinib, ruxolitinib, oclacitinib, peficitinib, fedratinib, delgocitinib, abrocitinib, deucravacitinib), anti-IFN antibodies (such as rontalizumab, sifalimumab, JNJ-55920839, AGS-009, Emapalumab), anti-IFN receptor antibodies (such as anifrolumab), bufexamac, SAR-20347. FLLL32 and vitamin E, or combinations thereof. In some embodiments, the preventing, interrupting, or alleviating comprises administering a composition comprising YM155. In some embodiments, the preventing, interrupting, or alleviating is mediated via delayed, targeted, or sustained release of or via chemically modified variants of said ILF3 inhibitor.

In an aspect, a method for treating pain in a subject includes inhibiting Interleukin Enhancer Binding Factor 3 (ILF3). In some embodiments, the type of pain is selected from the group consisting of: neuropathic pain, due to factors including but not limited to chemotherapy, nerve injury, nerve compression, autoimmune responses, retroviral infection: postoperative pain: myalgia: chronic pain: emergent pain: systemic pain; and pain associated with inflammation, infection, post-viral conditions (including, for example, myalgic encephalitis/chronic fatigue syndrome (ME/CFS) and post-acute sequelae of COVID-19 (PASC)), or disease. The preventing, interrupting, or alleviating comprises administering to the subject a polynucleotide, polypeptide, chemical compound, nanoparticles, delayed-release compound, or pharmaceutical.

In some embodiments, the treating comprises administering a compound selected from the group consisting essentially of: YM155, genistein, ivermectin, piroxicam, resveratrol, tamibarotene, thapsigargin, thimerosal, valproic acid, corticosteroids (such as prednisone, hydrocortisone, cortisone, methylprednisolone, dexamethasone, prednisolone), JAK/TYK inhibitors (such as baricitinib, tofacitinib, upadacitinib, ruxolitinib, oclacitinib, peficitinib, fedratinib, delgocitinib, abrocitinib, deucravacitinib), anti-IFN antibodies (such as rontalizumab, sifalimumab, JNJ-55920839, AGS-009, Emapalumab), anti-IFN receptor antibodies (such as anifrolumab), bufexamac, SAR-20347, FLLL32 and vitamin E, or combinations thereof. In some embodiments, the preventing, interrupting, or alleviating comprises administering a composition comprising YM155. In some embodiments, the preventing, interrupting, or alleviating is mediated via delayed, targeted, or sustained release of or via chemically modified variants of said ILF3 inhibitor.

In an aspect, the present disclosure provides methods for treating localized pain in a subject by inhibiting Interleukin Enhancer Binding Factor 3 (ILF3). In some embodiments, the type of pain is selected from the group consisting of: neuropathic pain, due to factors including but not limited to chemotherapy, nerve injury, nerve compression, autoimmune responses, retroviral infection: postoperative pain: myalgia: chronic pain: emergent pain: systemic pain; and pain associated with inflammation, infection, post-viral conditions (including, for example, myalgic encephalitis/chronic fatigue syndrome (ME/CFS) and post-acute sequelae of COVID-19 (PASC)), or disease. The preventing, interrupting, or alleviating comprises administering to the subject a polynucleotide, polypeptide, chemical compound, nanoparticles, delayed-release compound, or pharmaceutical.

In some embodiments, the treating comprises administering a compound selected from the group consisting essentially of: YM155, genistein, ivermectin, piroxicam, resveratrol, tamibarotene, thapsigargin, thimerosal, valproic acid, corticosteroids (such as prednisone, hydrocortisone, cortisone, methylprednisolone, dexamethasone, prednisolone), JAK/TYK inhibitors (such as baricitinib, tofacitinib, upadacitinib, ruxolitinib, oclacitinib, peficitinib, fedratinib, delgocitinib, abrocitinib, deucravacitinib), anti-IFN antibodies (such as rontalizumab, sifalimumab, JNJ-55920839, AGS-009, Emapalumab), anti-IFN receptor antibodies (such as anifrolumab), bufexamac, SAR-20347, FLLL32 and vitamin E, or combinations thereof. In some embodiments, the preventing, interrupting, or alleviating comprises administering a composition comprising YM155. In some embodiments, the preventing, interrupting, or alleviating is mediated via delayed, targeted, or sustained release of or via chemically modified variants of said ILF3 inhibitor.

In an aspect, the present disclosure provides a method for preventing pain induction, interrupting pain signaling, or alleviating pain perception in a subject by inhibiting the expression, activity, or function of Snail Family Transcriptional Repressor 1 (SNAI1) or Inhibin Subunit Alpha (INHA). In some embodiments, the type of pain is selected from the group consisting of: neuropathic pain, due to factors including but not limited to chemotherapy. nerve injury, nerve compression, autoimmune responses, retroviral infection: postoperative pain: myalgia: chronic pain: emergent pain: systemic pain; and pain associated with inflammation, infection, post-viral conditions (including, for example, myalgic encephalitis/chronic fatigue syndrome (ME/CFS) and post-acute sequelae of COVID-19 (PASC)), or disease. The preventing, interrupting, or alleviating comprises administering to the subject a polynucleotide, polypeptide, chemical compound, nanoparticles, delayed-release compound, or pharmaceutical.

### BRIEF DESCRIPTION OF FIGURES

FIG. 1. Viral Nucleocapsid protein (N) mRNA transcripts are acutely elevated in cervical dorsal root ganglia by quantitative polymerase chain reaction (qPCR). Φp<0.05 for two-way analysis of variance (ANOVA) interaction factor: *p<0.05, **p<0.01 for multiple t tests.

FIG. 2. N mRNA transcripts are acutely elevated in cervical spinal cord by qPCR. Φp<0.05 for two-way ANOVA interaction factor; *p<0.05, **p<0.01 for multiple t tests.

FIG. 3. N mRNA transcripts are acutely elevated in thoracic dorsal root ganglia by qPCR. Φp<0.05 for two-way ANOVA interaction factor: *p<0.05, **p<0.01 for multiple t tests.

FIG. 4. N mRNA transcripts are acutely elevated in thoracic spinal cord by qPCR. Φp<0.05 for two-way ANOVA interaction factor: *p<0.05, **p<0.01 for multiple t tests.

FIG. 5. Interferon-stimulated gene 15 (Isg15) mRNA transcripts are acutely elevated in cervical dorsal root ganglia by qPCR. Φp<0.05 for two-way ANOVA interaction factor: *p<0.05, **p<0.01 for multiple t tests.

FIG. 6. Isg15 mRNA transcripts are acutely elevated in cervical spinal cord by qPCR. Φp<0.05 for two-way ANOVA interaction factor; *p<0.05, **p<0.01 for multiple t tests.

FIG. 7. Isg15 mRNA transcripts are acutely elevated in thoracic dorsal root ganglia by qPCR. Φp<0.05 for two-way ANOVA interaction factor: *p<0.05, **p<0.01 for multiple t tests.

FIG. 8. Isg15 mRNA transcripts are acutely elevated in thoracic spinal cord by qPCR. Φp<0.05 for two-way ANOVA interaction factor: *p<0.05, **p<0.01 for multiple t tests.

FIG. 9. Plaque formation assay demonstrates mature virus presence only in the lungs of SARS-COV-2-infected hamsters at 3 days post infection (dpi), but not DRG or SC.

FIG. 10. Mechanical thresholds of mock, influenza A virus (IAV), and SARS-CoV-2 animals at baseline, 1 dpi, and 4 dpi. IAV induced severe hypersensitivity at 1 dpi, and SARS-COV-2 induced mild hypersensitivity by 4 dpi (n=4 per group: *p<0.05 for one-way ANOVA Tukey's m.c.). IAV infection resulted in significantly lower thresholds than SARS-CoV-2 on Idpi (Φp<0.05 for two-way ANOVA Tukey's multiple comparison test (“Tukey's m.c.”).

FIG. 11. Petal diagrams for 1 dpi and 4 dpi SARS-COV-2 and IAV tDRG Differentially Expressed Genes (DEGs) (p adj.<0.01), with commonly upregulated or downregulated genes.

FIG. 12. Top 5 IPA Canonical Pathways for 1 dpi SARS-COV-2 tDRGs vs Mock (p-nom.<0.05:-log 10 (p-value)>1.3).

FIG. 13. Top 5 IPA Canonical Pathways for 1 dpi IAV tDRGs (p-nom.<0.05;-log 10 (p-value)>1.3).

FIG. 14. Top 5 IPA Canonical Pathways for 4 dpi SARS-COV-2 tDRGs (p-nom.<0.05:-log 10 (p-value)>1.3).

FIG. 15. Top 5 IPA Canonical Pathways for 4 dpi IAV tDRGs (p-nom.<0.05;-log 10 (p-value)>1.3).

FIG. 16. qPCR validation of 1 dpi and 4 dpi SARS-COV-2 tDRG DEGs (*p<0.05, ****p<0.0001 for multiple t-tests)

FIG. 17. IPA predicted upstream regulators that had predicted inhibition in 1 and 4 dpi SARS-COV-2 tDRG and 4 dpi IAV DEGs (*Benjamini-Hochberg p<0.05).

FIG. 18. No significant changes in Ilf3 gene expression were observed in SARS-CoV-2, IAV, or Mock tDRGs 1 and 4 dpi in accordance with sequencing.

FIG. 19. IPA prediction of ILF3-regulated genes at 1 and 4 dpi in SARS-COV-2 tDRGs.

FIG. 20. YM155 (5 mg/kg i.p. QD) increased thermal thresholds 30-60 minutes after administration (*p<0.05, ****p<0.0001 for two-way ANOVA Sidak's m.c.).

FIG. 21. YM155 (5 mg/kg i.p. QD) increased mechanical thresholds 30-60 minutes after administration (*p<0.05, ****p<0.0001 for two-way ANOVA Sidak's m.c.).

FIG. 22. YM155 (5 mg/kg i.p. QD) increased thermal thresholds in a sustained fashion ˜24 hours after administration by 6 days after initial administration (*p<0.05, **p<0.01, ***p<0.001 for two-way ANOVA Sidak's m.c.).

FIG. 23. YM155 (5 mg/kg i.p. QD) increased mechanical thresholds in a sustained fashion ˜24 hours after administration by 5 days after initial administration (*p<0.05, **p<0.01, ***p<0.001 for two-way ANOVA Sidak's m.c.).

FIG. 24. No changes in post-CFA weight were observed in YM155 (5 mg/kg i.p. QD) animals throughout the course of administration.

FIG. 25. Concurrent treatment with YM155 (5 mg/kg i.p. QD) in female mice that received a hindpaw injection of IFNβ (300U/25 μL) interrupted the onset of interferon-induced mechanical hypersensitivity (****p<0.0001 for two-way ANOVA Sidak's m.c.).

FIG. 26. Pre-treatment with YM155 (5 mg/kg i.p. QD) led to significantly lower mechanical hypersensitivity after paw incision (*p<0.05 for two-way ANOVA Sidak's m.c.).

FIG. 27. No differences in locomotion were observed on Day 1 post-paw incision between YM155 and Saline mice.

FIG. 28. Mechanical thresholds of mock, IAV, and SARS-COV-2 animals at 28 dpi (**p<0.01, ****p<0.0001 for one-way ANOVA Tukey's m.c.).

FIG. 29. IPA top 10 canonical pathways (−log 10 (p-value)>1.3) associated with 31 dpi SARS-COV-2 tDRG DEGs (p-nom.<0.05).

FIG. 30. Enrichr DisGeNET gateway top 10 diseases associated with 31 dpi SARS-COV-2 tDRG DEGs (p-nom<0.05).

FIG. 31. Log 2 (FC) of select neuronal and inflammatory genes from 31 dpi RNA-seq (p-adj.<0.1).

FIG. 32. Positively and negatively enriched cell subtypes associated with 31 dpi SARS-COV-2 tDRG DEGs (GSEA Net Enrichment Score>|1.5|; DEG p-adj.<0.1).

FIG. 33. IPA top 15 upstream regulators between 31 dpi SARS-COV-2 tDRG, Striatum, and Thalamus (DEG p-nom.<0.05).

FIG. 34. Chord diagrams demonstrating regulation of DRG gene expression changes between complete Freund's adjuvant (CFA), spared nerve injury (SNI), and SARS-CoV-2 (1, 4, and 31 dpi) animals.

FIG. 35. Dot plots demonstrating significant Gene Ontology (GO: Molecular Function, Biological Process, Cellular Compartment), Kyoto Encyclopedia of Genes and Genomes (KEGG), Reactome (REAC), WikiPathways (WP), Transfac (TF), and Human Protein Atlas 686 (HPA) (−log 10p-adj.>1.3) for contra-regulated genes between SNI and 1 dpi SARS-COV-2, conserved upregulated genes for CFA/SNI vs 1˜4 dpi SARS-COV-2 comparisons, and all commonly regulated genes between SNI & 31 dpi SARS COV-2. Dot plots adapted from g: Profiler.

### DETAILED DESCRIPTION

### Definitions

While various embodiments of the disclosure have been shown and described herein, those skilled in the art will understand that such embodiments are provided by way of example only. Numerous variations, changes, and substitutions may occur to those skilled in the art without departing from the disclosure. It would be understood by a person of skill that various alternatives to the embodiments of the disclosure described herein may be employed.

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

The term “subject,” as used herein, generally refers to an animal, such as a mammalian species {e.g., human) or avian {e.g., bird) species, or other organism, such as a plant. More specifically, the subject can be a vertebrate, e.g., a mammal such as a mouse, a primate, a simian or a human. Animals include, but are not limited to, farm animals, sport animals, and pets. A subject can be a healthy individual, an individual that has or is suspected of having a disease or a pre-disposition to the disease, or an individual that is in need of therapy or suspected of needing therapy. A subject can be a patient.

As used herein the term “ILF3” refers to Interleukin Enhancer Binding Factor 3.

### Overview

Utilizing the established golden hamster model of COVID-19, the inventors detected low levels of SARS-COV-2-derived viral RNA in the absence of infectious particles. The inventors discovered that exposure of sensory tissues to this viral material and/or the resulting type I interferon response correlated with a progressive and prolonged mechanical hypersensitivity signature that was unique to SARS-COV-2. Transcriptomic analysis of thoracic SARS-COV-2 RNAemic DRGs surprisingly revealed a pronounced neuronal signature unlike the predominantly pro-inflammatory signature seen in DRGs from animals infected with Influenza A Virus (IAV). The inventors discovered that SARS-COV-2 infection also correlated with increased hypersensitivity post-recovery in both female and male hamsters. Transcriptional profiling of thoracic DRGs at 1, 4, and 31 dpi implicated several therapeutic targets for the management of chronic pain. The inventors confirmed ILF3 inhibition as a target for therapeutic intervention in two murine hindpaw injection models-CFA for a combined interferon and cytokine response and IFN-B for an isolated type I interferon response that mimics myalgia, as well as the paw incision model, which represents local inflammation and small sensory fiber damage after surgery. Finally, meta-analysis against existing transcriptional data sets from pain models highlighted several unexplored acutely and chronically contra-regulated genes between SARS-COV-2 and SNI that could serve as future targets for anti-nociceptive therapies and provide novel mechanistic insight into these perturbations.

The SARS-COV-2 RNA infiltration dynamics within sensory tissue observed by the inventors were similar to those noted in their longitudinal study of SARS-COV-2 effects on the brain, where a rapid transcriptional induction to infection is followed by a return to baseline in most, but not all tissues. While the inventors confirmed the presence of SARS-COV-2 RNA in various cell types of the DRG, they were surprised by the neuronally-biased transcriptional responses associated with this positivity that were not as prominent in tissue from IAV-infected hamsters.

Few pain therapeutics target both the peripheral and central site of the nociceptive pathway. Here, the inventors identified that several common predicted upstream regulator (UR) targets exist between the DRGs and brain regions that process pain and emotion. While most of the top common URs were counter-regulated between the DRGs and Thalamus/Striatum, three pain- and affect-associated URs (PTPRR, miR17HG, and FIRRE) were predicted to change unidirectionally between DRG and Thalamus. Prior work performed by the inventors has shown a high level of treatment effectiveness in targeting the same protein in DRG and Thalamus through studies on the signal transduction modulator RGS4, which has a pro-allodynic/hyperalgesic role in these regions. Notably, the present disclosure shows that the expression of the Rgs4 gene was decreased in Idpi in DRGs of SARS-COV-2-infected hamsters, which is potentially a strategy by which acute SARS-COV-2 infection leads to less myalgic hypersensitivity than other viruses, such as IAV.

While some reports have recapitulated human COVID-19 symptoms in the present respiratory hamster model, this is the first study that confirmed the model's relevance for somatosensory symptoms. The present disclosure reveals that this model accurately aligns with the somatosensory trajectory of many COVID-19 patients, both acutely and chronically. This SARS-COV-2 model is also useful for further identifying core mechanisms across pain models, while also providing insights into novel viral mediated nociceptive states with relevance for drug development.

In addition to elucidating the impact SARS-COV-2 has on DRGs, the inventors also identified a subset of host factors as modulators of the nociceptive responses. Notably, increased activity of ILF3 is generally considered oncogenic. Furthermore, several of the disease risk signatures associated with gene changes observed in the 31 dpi SARS-COV-2 DRGs revolved around neuronal and glial cancers. Given the inventors' current (ILF3 inhibitor) and prior (Rgs4 downregulation, HDAC1 inhibition, and HDAC6 inhibition) successes with use of cancer-targeting therapies for the treatment of inflammatory and nerve injury-associated pain states, they decided to repurpose existing clinically-validated cancer therapeutics, such as YM155, to provide alternative treatments for pain management.

### EXAMPLES

### Example 1: SARS-COV-2 RNA Infiltrates Thoracic and Cervical DRG and Spinal Cord Tissue

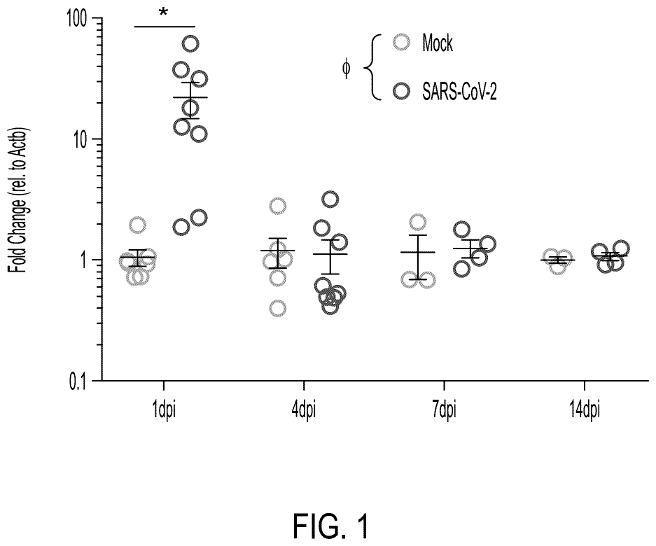

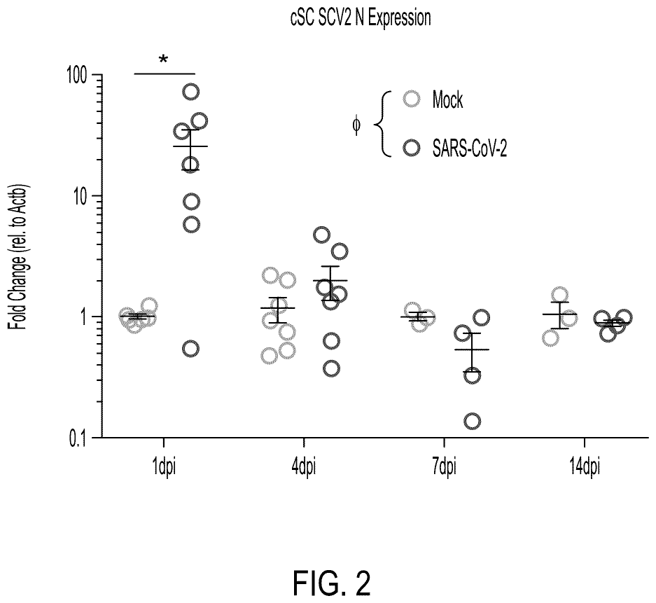

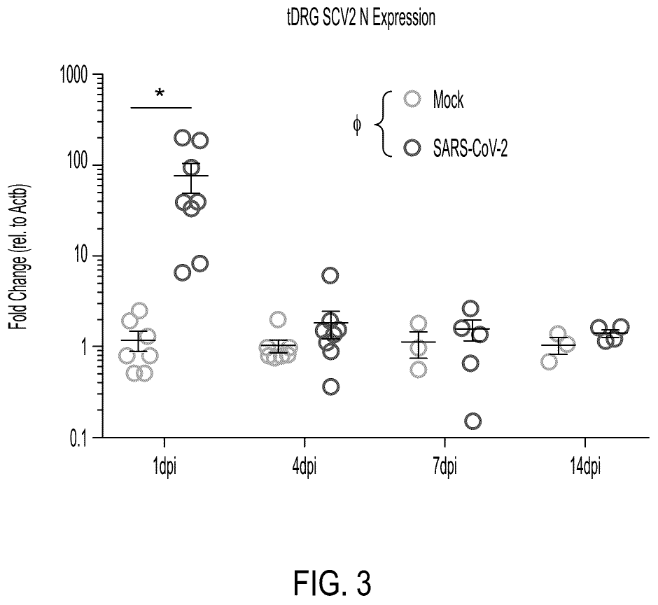

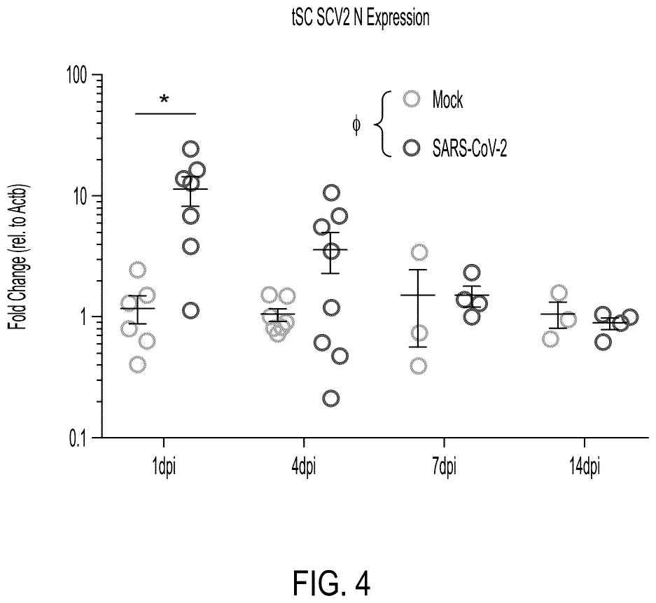

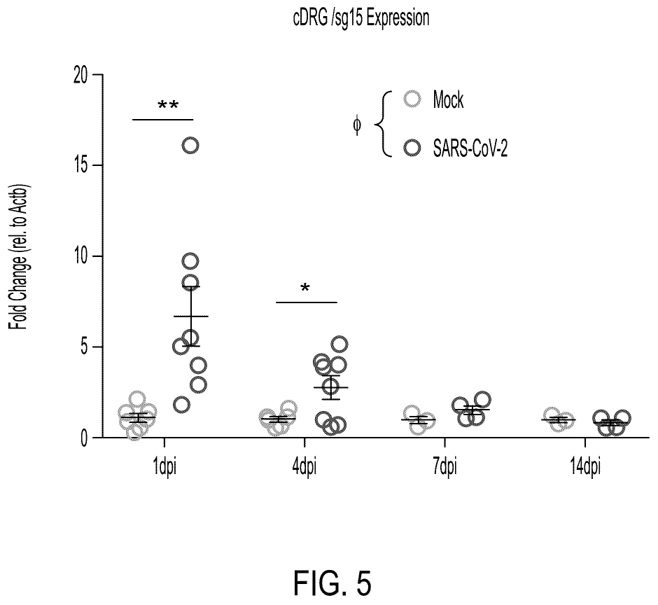

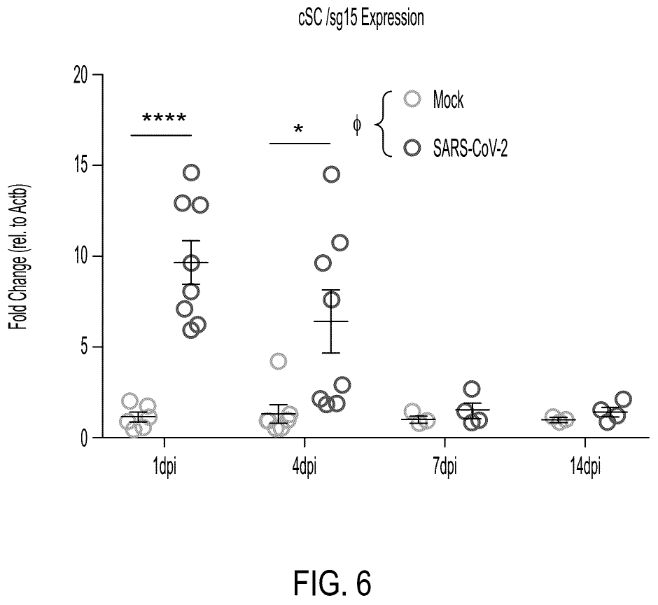

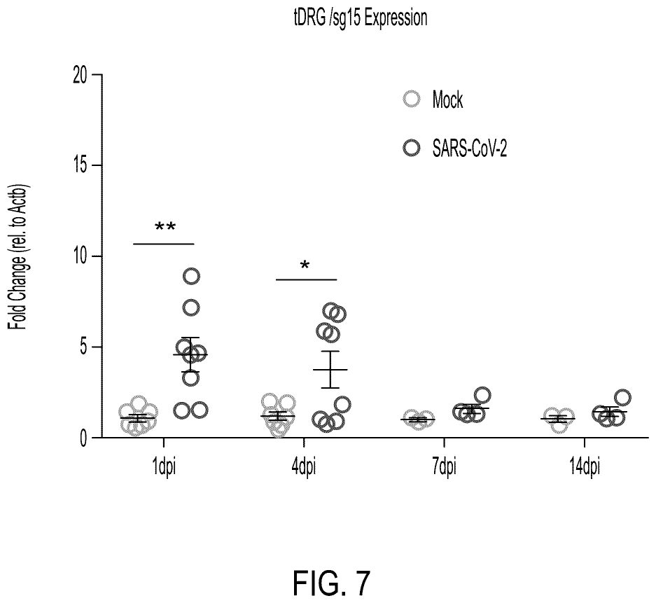

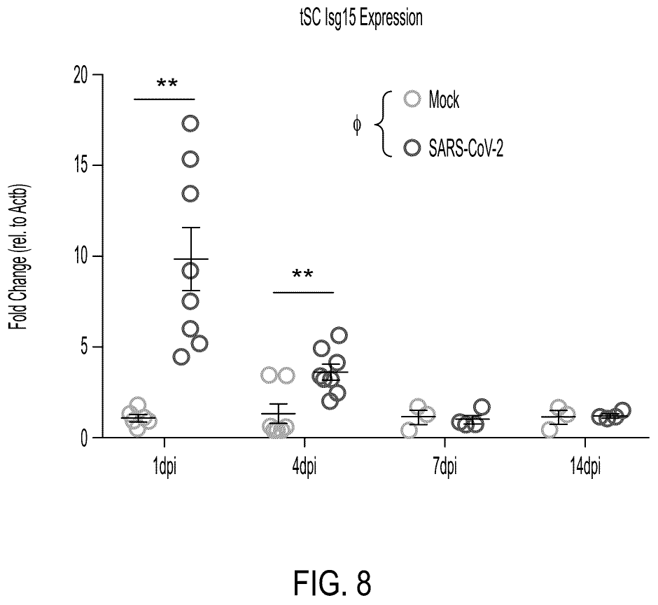

The inventors first examined whether SARS-COV-2 genetic material is present in the sensory nervous system tissues and investigated if this presence was associated with induction of an antiviral response. To this end, the inventors performed a longitudinal cohort study in which hamsters were treated intranasally with SARS-COV-2 or PBS (mock-infected). Cervical and thoracic levels of DRGs and spinal cord were harvested at 1, 4, 7, and 14 dpi in both groups and assessed for the presence of SARS-COV-2 subgenomic nucleocapsid protein (N) and canonical type-I interferon stimulated gene Isg15 transcripts via quantitative reverse transcription PCR (RT-qPCR). The inventors discovered a substantial elevation of nucleocapsid protein (N) transcripts at Idpi in cervical DRGs (FIG. 1: two-way ANOVA interaction F(3,35)=4.205, p=0.0122: multiple t-tests 1 dpi t=2.698, df=13, p=0.0183), cervical SC (FIG. 2: two-way ANOVA interaction F(3,35)=3.809, p=0.0189: multiple t-tests 1 dpi t=2.392, df=11, p=0.0358), thoracic DRGs (FIG. 3: two-way ANOVA interaction F(3,36)=3.812, p=0.018: multiple t-tests Idpi, t-2.528, df=13, p=0.0252), and thoracic SC (FIG. 4: two-way ANOVA interaction F(3,34)=4.266, p=0.0116; multiple t-tests 1 dpi t=3.068, df=11, p=0.0107). Viral RNA appeared to be cleared in most samples by 4 dpi. Isg15 mRNA levels, which are generally representative of interferon signaling, had similar elevation patterns to those of N in cervical DRGs (FIG. 5: two-way ANOVA interaction F(3,35)=3.689, p=0.0208, multiple 147 t-tests 1 dpi t=3.152, df=13, p=0.00764: 4 dpi t=2.361, df=12, p=0.0360), cervical SC (FIG. 6: two-way ANOVA interaction F(3,35)=5.001, p=0.0054; multiple t-tests Idpi t=6.034, df=12, p=0.0000590; 4 dpi t=2.656, df=13, p=0.0198), thoracic DRGs (FIG. 7: two-way ANOVA interaction F(3,36)=1.856, p=0.155: multiple t-tests 1 dpi t=3.541, df=13, p=0.00362: 4 dpi t=2.311, df=13, p=0.0379), and thoracic SCs (FIG. 8: two152way ANOVA interaction F(3,35)=8.478, p=0.0002: multiple t-tests 1 dpi t=4.286, df=12, p=0.00106: 4 dpi t=3.333, df=13, p=0.00539).

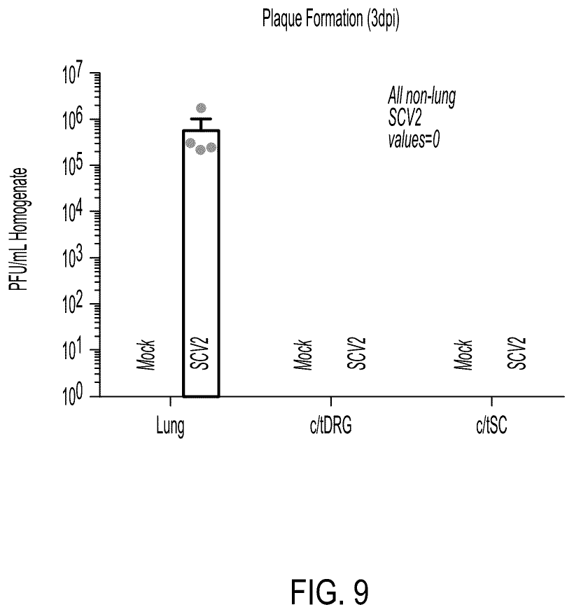

To gain insight into viral replication within the DRG, the inventors performed a plaque assay in which combined cervical and thoracic DRGs or SC were collected at 3 dpi and homogenized in PBS. This solution was then plated with Vero cells, with the number of ensuing plaques representing the number of mature virions present in the harvested tissue. As seen in FIG. 9, plaques were observed only in 3 dpi lung homogenate from SARS-COV-2-infected animals, but not in mock lung or any DRG or SC tissue. This revealed that mature virus was not reaching the peripheral or central sensory nervous systems.

The inventors next sought to determine whether SARS-COV-2 transcripts were localized to specific cell types in the DRG, which is predominantly composed of primary sensory neurons and satellite glial cells. By using RNAscope in situ hybridization on Idpi cervical and thoracic cell tissue, the inventors observed the presence of SARS-COV-2 spike protein transcript(S) puncta around DAPI-labeled nuclei, which in DRGs are representative of satellite glial cells and Rbfox3-labeled neuronal spaces, but not in mock samples. The inventors also detected S transcript puncta near DAPI signal throughout SARS-COV-2-infected cervical and thoracic spinal cord sections on Idpi, but not in mock samples (FIG. 2B).

Notably, when tissue sections obtained from the DRGs of SVC2- or mock-infected hamsters were immuno-labeled for SARS-COV-2 nucleocapsid protein (NP) the inventors did not observe any notable viral protein presence. Importantly, the inventors confirmed the presence of NP in SARS-COV-2-infected lung samples, but not in mock controls. This raised the question of whether the presence of viral RNA and associated antiviral response signatures in the sensory nervous system are sufficient to induce behavioral and/or transcriptional perturbations.

### Example 2: SARS-COV-2 and IAV Induce Unique Mechanical Hypersensitivity Signatures

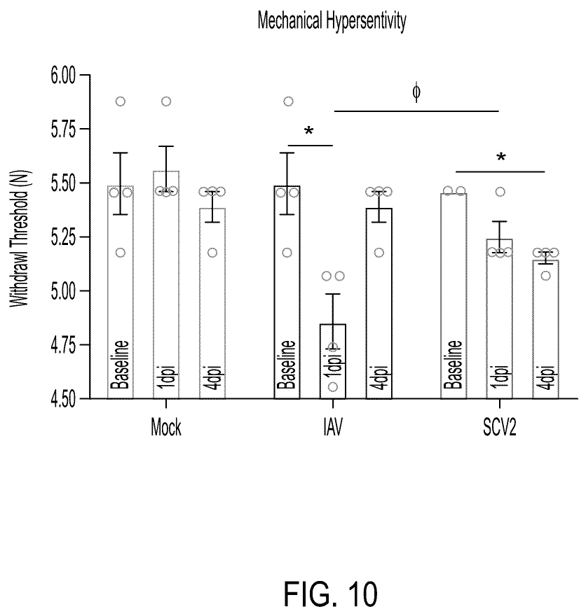

The inventors next sought to determine whether the presence of SARS-COV-2 RNA or associated type I interferon (IFN-I) signaling, as reported previously, was associated with the induction of sensory hypersensitivity. To assess this, the inventors performed the von Frey assay on hamsters infected with either IAV (A/California/04/2009) or SARS-COV-2. IAV is an RNA virus of the respiratory tract that is known to provoke a systemic inflammatory response resulting in clinically-associated myalgias, similar to SARS-COV-2. von Frey thresholds were measured during the acute phase of infection (1 and 4 dpi) to identify the effects of active and subsiding SARS-COV-2 mRNA presence and IFN-I response on sensation. As seen in FIG. 10, the inventors observed a significant interaction effect between time and virus on mechanical hypersensitivity (RM two-way ANOVA Interaction F(4,18)=4.16, df=4, p=0.0147). IAV induced robust hypersensitivity at Idpi which completely subsided by 4 dpi (one-way ANOVA F=6.092, p=0.0359; Tukey's m.c.: Baseline vs. 1 dpi q=4.604, df=6, p=0.0398). SARS-COV-2 infection instead resulted in a gradual exacerbation of hypersensitivity, reaching significance only at 4 dpi (one-way ANOVA F=9.772, p=0.013: Tukey's m.c.: Baseline vs. 4 dpi q=6.117, df=6, p=0.0117). Importantly, Idpi IAV-induced hypersensitivity was significantly higher than that caused by Idpi SARS-COV-2 (RM two-way ANOVA Tukey's m.c. q-4.033, df-27, p=0.0218). Considering the emergence of distinct behavioral signatures irrespective of systemic interferon responses induced by these two viruses, the inventors performed a time-dependent transcriptional comparison of sensory structures after infection.

### Example 3: Sensory Transcriptional Response to SARS-COV-2 Infection

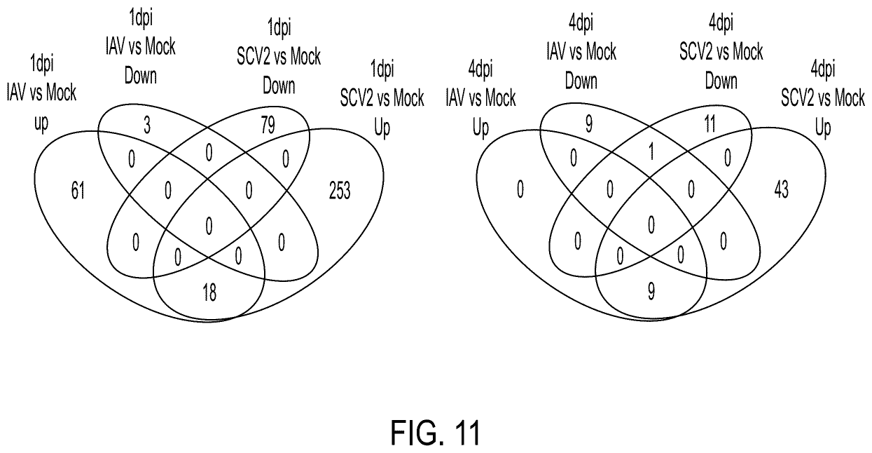

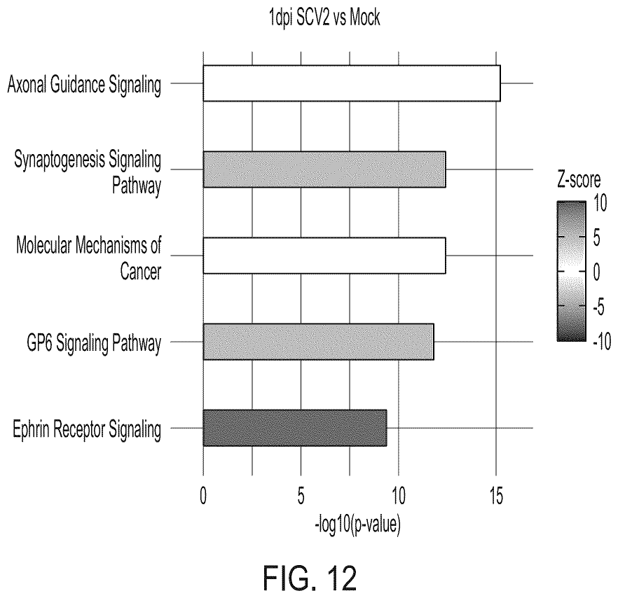

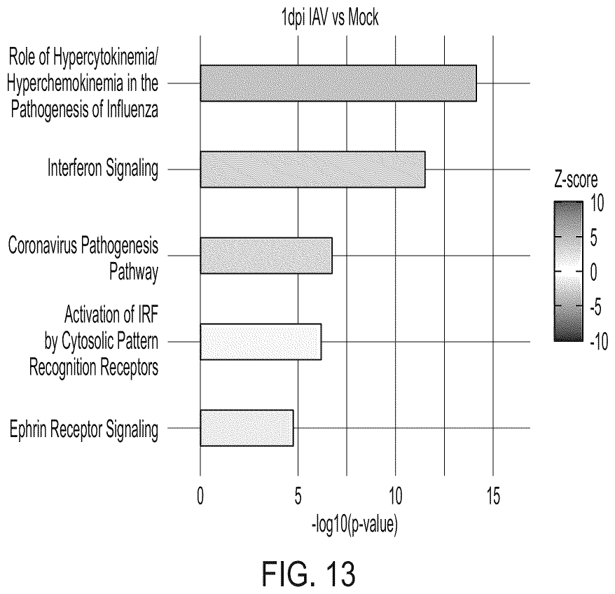

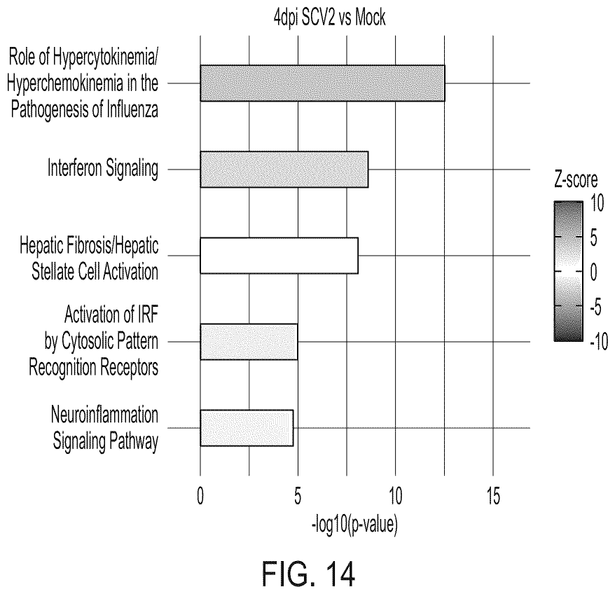

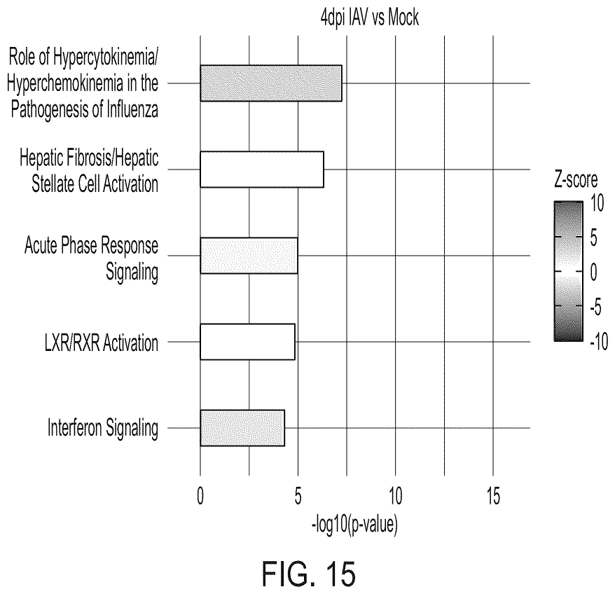

The inventors conducted transcriptional profiling via RNA-seq on thoracic DRGs from SARS-COV-2- and IAV-infected hamsters at both Idpi and 4 dpi because of their respiratory, visceral, and dermal innervations. Differential expression analysis of RNA-seq data revealed transcriptomic changes in both SARS-COV-2- and IAV-infected thoracic DRGs compared to mock at 1 dpi and 4 dpi. SARS-COV-2 infection resulted in a more robust differential expression at both time points: 344 genes at Idpi (271 up & 79 down: p-adj.<0.1) and 63 genes at 4 dpi (52 up & 11 down: p adj.<0.1). IAV infection resulted in differential expression of 82 genes at 1 dpi (79 up & 3 down: p-adj.<0.1) and 18 genes at 4 dpi (9 up & 9 down: p-adj.<0.1) (FIG. 11). Considering the milder acute mechano-sensitivity phenotype in SARS-COV-2-infected hamsters and greater differential gene expression compared to IAV-infected hamsters, the inventors hypothesized that certain acute SARS-COV-2-induced transcriptional changes may counteract interferon-induced somatosensory sensitization, potentially by causing a stronger neuronal gene adaptation signature. To better assess this, the inventors performed a canonical pathway analysis (IPA, Qiagen) on their RNA-seq data. This analysis showed neuron-specific transcriptional differences within the reported top upregulated canonical pathways (based on genes with nominal p<0.05) (FIGS. 12-14). The top two most enriched pathways for Idpi SARS-COV-2 tissue was “Axonal Guidance Signaling” and “Synaptogenesis Signaling”, and at 4 dpi “Neuroinflammation Signaling” was among the top-five pathways. However, for IAV samples, the top canonical pathway results were consistently representative of generic viral response pathways.

To better understand which transcripts were driving these enriched annotations, the inventors compared DEGs (p-adj.<0).1) between tissues derived from IAV- and SARS-COV-2-infected hamsters. Commonly upregulated genes between Idpi and 4 dpi SARS-COV-2 and IAV tissues were primarily anti-viral in nature, with only one co-downregulated gene emerging at 4 dpi, Svep1 (a vascular gene whose locus has been associated with poor SARS-COV-2 clinical outcomes). RNA-seq was validated at Idpi and 4 dpi through qPCR measurement of neuronal and anti-viral genes from SARS-COV-2 and mock tissues.

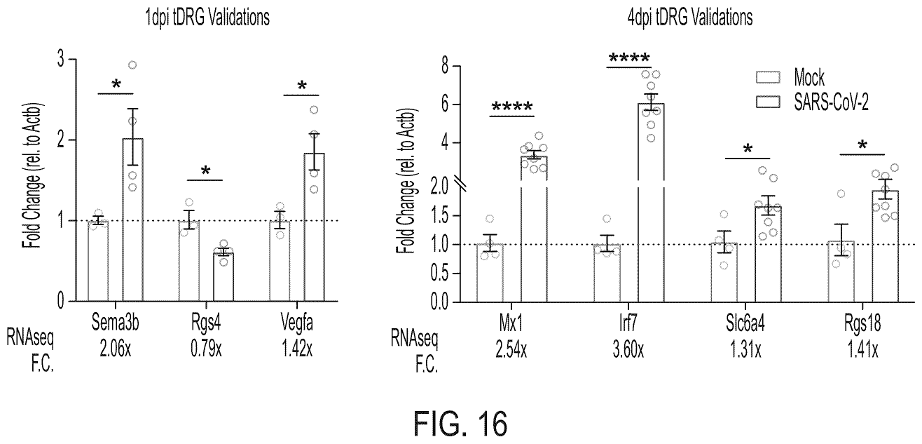

Surprisingly, the inventors observed bi-directional regulation of neuropathy-associated and/or pro-nociceptive genes at Idpi, such as upregulation of Sema3b and Vegfa and downregulation of Rgs4 (FIG. 16). qPCR validations of 4 dpi included upregulation of Mx1 and Irf7 (pro-inflammatory, anti-viral genes), as well as Slc6a4 and Rgs18, which have also been implicated in sensory abnormalities (FIG. 16).

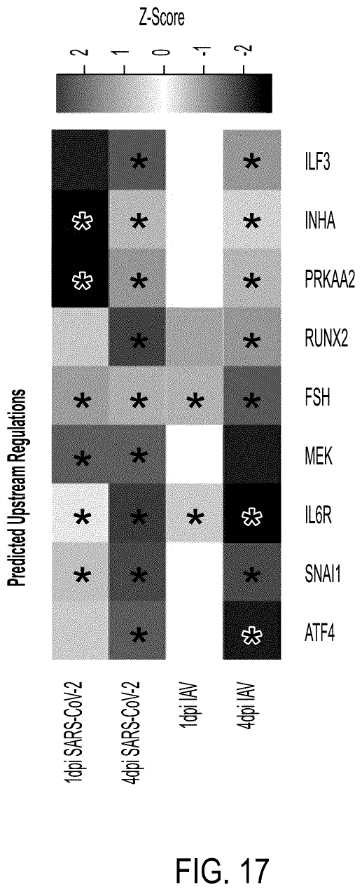

Analysis of upstream regulators (URs: IPA. Qiagen) of differentially expressed nominal p<0.05 genes on IPA revealed several commonly- and oppositely-regulated URs between SARS-COV-2 and IAV datasets. Based on their hypothesis that SARS-COV-2 transcriptionally counteracts interferon-induced hypersensitivity, the inventors sought to identify URs uniformly associated with timepoints of acute viral infection during which lower levels of hypersensitivity were observed, namely Idpi SARS-COV-2, 4 dpi SARS-COV-2, and 4 dpi IAV. They focused on URs with predicted downregulated activity to find inhibition targets. Nine URs met this criterion: Interleukin 6 Receptor (IL6R). Mitogen-activated Protein Kinase (MEK), Interleukin Enhancer-binding Factor 3 (ILF3). Runt-related Transcription Factor 2 (RUNX2). Protein Kinase AMP-Activated Catalytic Subunit Alpha 2 (PRKAA2) (UR was AMPKα2 gene). Follicle Stimulating Hormone (FSH). Activating Transcription Factor 4 (ATF4), Snail Family Transcriptional Repressor 1 (SNAI1), and Inhibin Subunit Alpha (INHA) (FIG. 17). Interestingly, pre-clinical and clinical literature supports a positive association between upregulation/activation of IL6R. MEK. RUNX2. FSH. & ATF4 and nociceptive states, and several laboratories have validated interventions in relevant pathways as promising anti-nociceptive therapeutic strategies. Only AMPKα2 activity was expressed towards a pro-nociceptive direction in this list, as pre-clinical literature suggests activation of this protein is associated with the alleviation of nociceptive symptoms. These data suggest that other targets in this list may serve as novel therapeutic avenues of pain. Among the identified 247 genes that have not been studied in pain models (SNAI1. ILF3, and INHA), the inventors selected to study ILF3 and determine whether a commercially available inhibitor. YM155, could affect pain perception or signaling.

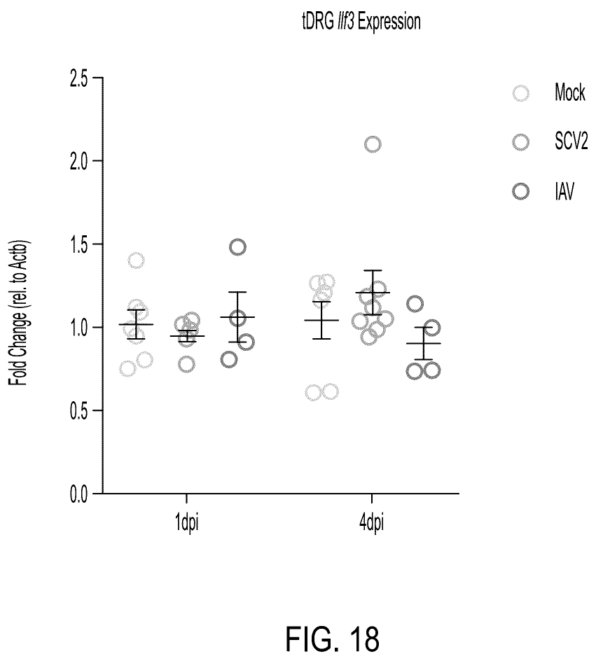

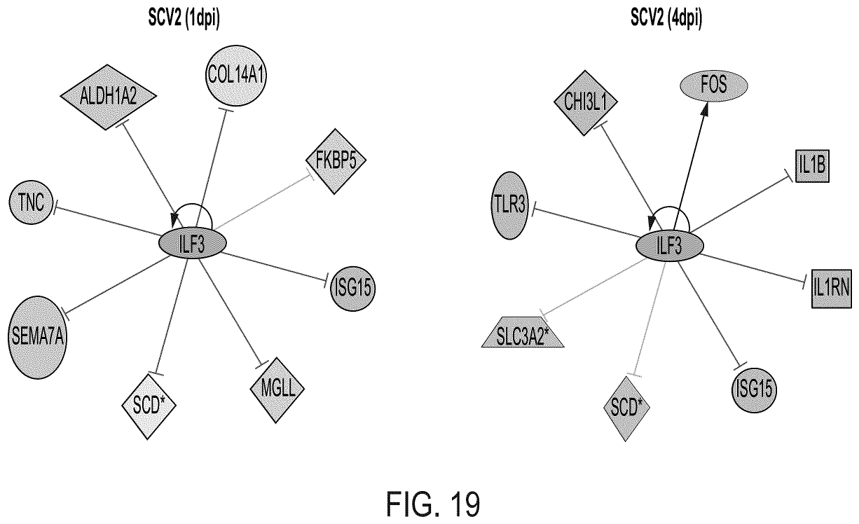

Based on predicted interactions between ILF3 and SARS-COV-2-regulated genes, the inventors investigated it as a pain target, particularly since prior research has revealed several genes to be associated with either neuronal activity/plasticity (including Fos. Col14a1, Aldh1a2, Fkbp5, Sema7a, Mgll Chi3l1, and Slc3a2) or with interferon and cytokine responses (including Isg15, Il1b, Il1m, Tlr3. Tnc) (FIG. 19). As the inventors predicted based on RNAseq results (which did not label Ilf3 as a significant DEG, but rather one whose activity might be altered by SARS-COV-2), whole tissue qPCR demonstrated a lack of Ilf3 gene expression changes in SARS-COV-2 and IAV tissues from 1 dpi or 4 dpi timepoints, suggesting that changes in the activity of this molecule are occurring at the protein level (FIG. 18). The inventors noted that YM155 is predicted to affect subcellular localization of ILF3 and its associated complexes, as opposed to directly inhibiting its expression.

### Example 4: Inhibition of ILF3 Activity Alleviates Sensory Hypersensitivity in an Inflammatory Pain Model

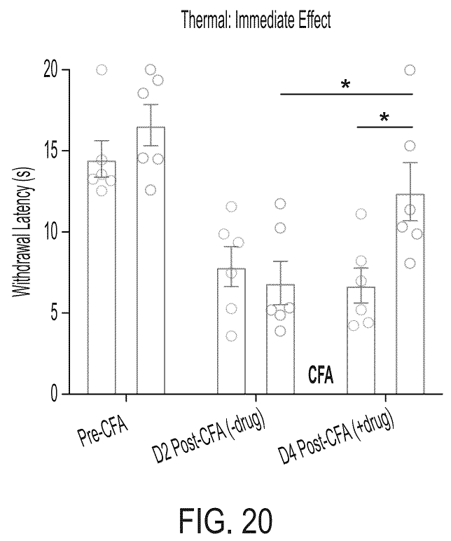

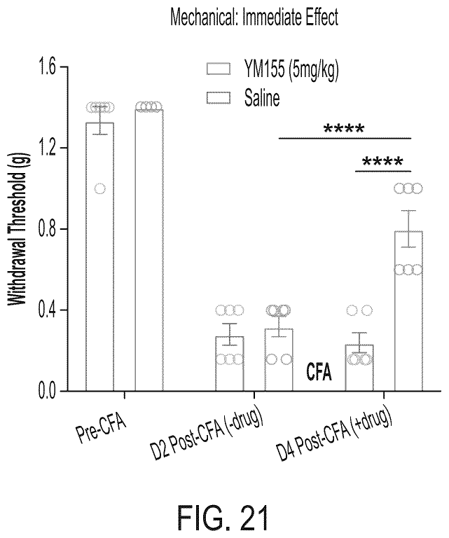

The inventors used the CFA model of peripheral inflammation in female mice to determine the impact of ILF3 inhibition in sensory hypersensitivity behaviors associated with inflammatory pain states. The inventors observed lethal toxicity at 20 mg/kg, so they proceeded with a 5 mg/kg once-daily regimen. To identify any immediate analgesic effects of YM155 under local, peripheral inflammation conditions, the inventors first tested CFA-injected mice in the Von Frey and Hargreave's assays at 30 minutes post-drug administration. The inventors discovered that YM155-treated mice displayed increased Hargreaves' response times (FIG. 20; RM two-way ANOVA Interaction: F(2,20)=4.116, df=2, p=0.0318: Sidak's m.c.: YM155 vs Saline D3 Post-CFA (+drug) t=3.085, df=30, p=0.013: YM155 D2 Post-CFA (−drug) vs D3 Post-CFA (+drug) t=3.36, df-20, p=0.0186) and increased von Frey thresholds (FIG. 21: RM two-way ANOVA Interaction: F(2,20)=13.5, df=2, p=0.0002; Sidak's m.c.: YM155 vs Saline Day 4 Post-CFA (+drug) t=6.784, df=30, p<0.0001, YM155 D2 Post-CFA (−drug) 272 vs D4 Post-CFA (+drug) t=8.517, df=20, p<0.0001).

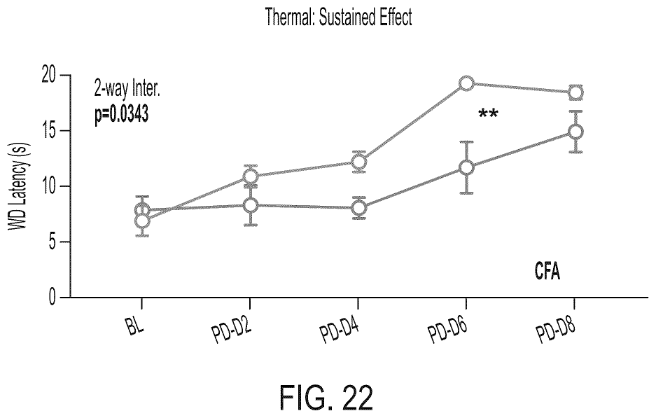

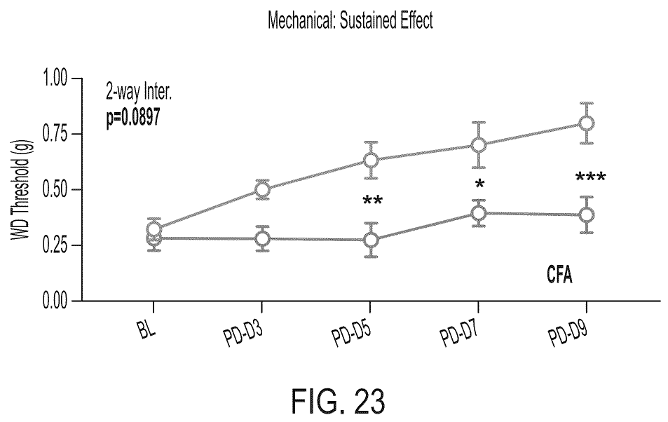

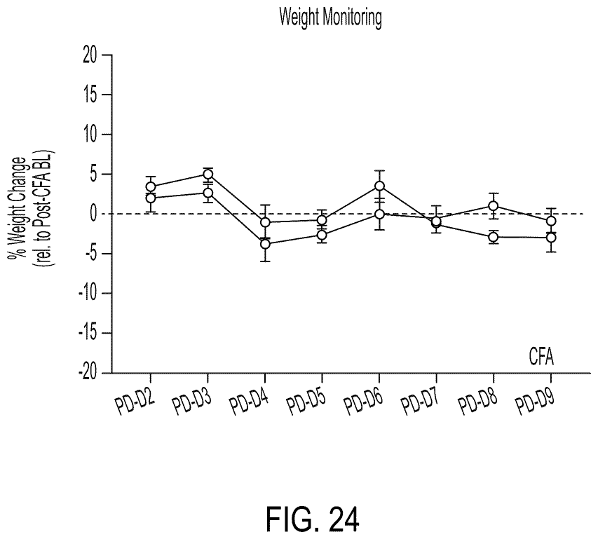

The inventors also tested whether YM155 had sustained effects on sensory hypersensitivity after the expected window of activity (approximately 24 hours post-injection, based on a ˜one hour half-life in intravenously-treated mice). Surprisingly, when mice were monitored in the Hargreave's assay at 24 hours post-injection, the inventors observed a significantly higher withdrawal latency at six consecutive days (PD-D6) of YM155 administration (FIG. 22: RM two-way ANOVA Interaction: F(4,40)=2.887, df=4, p=0.0343; Sidak's m.c.: YM155 vs Saline PD-D6 t=3.964, df=50, p=0.0012), prior to the expected recovery from thermal hypersensitivity in CFA animals. Similarly, the inventors observed sustained recovery of mechanical thresholds on PD-D5, PD-D7, and PD D9 in the Von Frey assay (FIG. 23: RM two-way ANOVA Interaction: F(4,40)=2.171, df=4, p=0.0897: Sidak's m.c.: YM155 vs Saline PD-D5 t=3.59, df=50, p=0.0038; PD-D7 t=3.058, df=50, p=0.0177; PD-D9 t=4.122, df=50, p=0.0007). No changes in weight due to YM155 administration were observed over the first 9 days of treatment (FIG. 24).

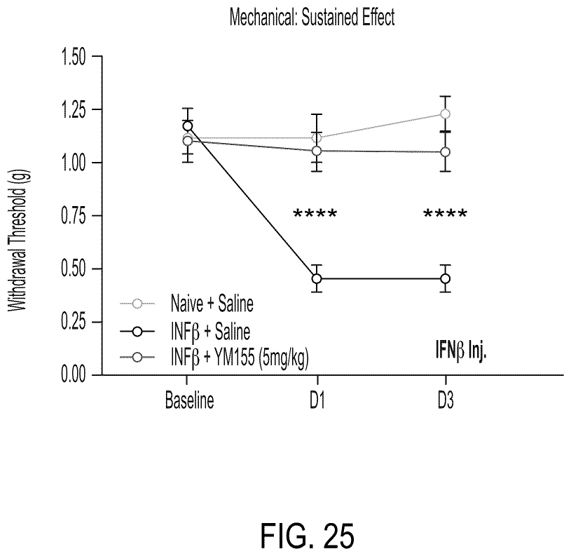

While the CFA model promotes both peripheral interferon and cytokine signaling. the inventors wanted to investigate the role of ILF3 inhibition in a model of isolated interferon signaling to better understand its role in virus-induced hypersensitivity. For this, the inventors implemented hindpaw interferon β (IFNβ) injections. The inventors compared mechanical thresholds between a group of female mice that received no IFNβ injection and daily saline (Naïve-Saline), a group that received a single IFNβ injection and daily saline (IFNβ-Saline). and a group that received a single IFNβ injection and immediate daily administration of YM155 (5 mg/kg i.p. BID: IFNβ-YM155). On days 1 and 3 after IFNβ administration, the inventors discovered robust mechanical hypersensitivity in the IFNβ-Saline group, but no change in thresholds in the IFNβ-YM155 group, suggesting that ILF3 inhibition attenuates interferon-induced hypersensitivity (r.m, two-way ANOVA Interaction Factor F(4.38)=17.33, p<0.0001: Sidak's m.c. IFNβ-Saline vs IFNβ-YM155 Day 1 t=4.94, df=57, p<0.0001: Day 3 t=4.94. df=57, p<0.0001) (FIG. 25). Surprisingly, when testing YM155 in male mice, the inventors noted increased sensitivity to the drug and subsequent toxicity at the 5 mg/kg dose. The inventors therefore performed an experiment in which a Naïve-Saline and IFNβ-Saline group began receiving YM155 (2.5 mg/kg i.p. BID) after mechanical thresholds were taken one day after intraplantar injections. At this dose, the inventors did not observe toxicity nor changes in the Naïve group's withdrawal thresholds, whereas they discovered a substantial increase in mechanical thresholds in the IFNβ group by day three after intraplantar injections (r.m, two-way ANOVA Interaction Factor F(2.24)=11.17, p=0.0004). This demonstrates that, while sex-specific dose optimization is necessary with YM155. ILF3 inhibition is effective at suppressing interferon-induced hypersensitivity in both mouse sexes.

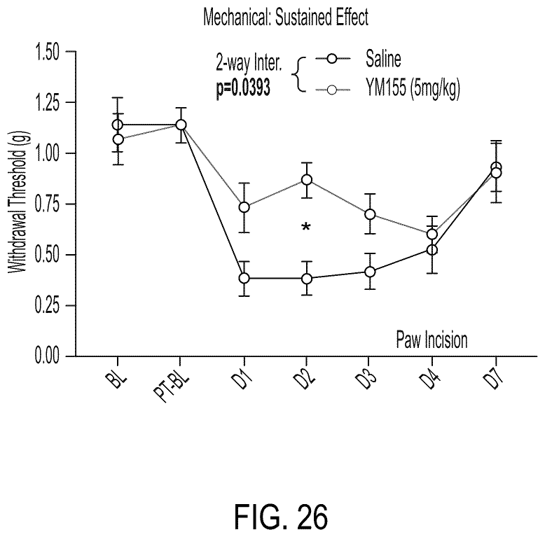

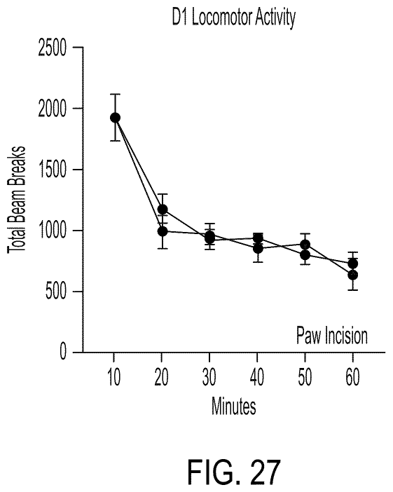

The inventors also tested whether YM155 could be used to prophylactically reduce pain experienced after acute post-operative injuries. For this, the inventors used the paw incision model and pre-treated animals at a dose of 5 mg/kg i.p, for seven days. Animals were not treated with drug after the incision. The inventors discovered a significant reduction in mechanical hypersensitivity due to the incision (FIG. 26; RM two-way ANOVA Interaction: F(6.60)=2.384, df=6, p=0.0393; Sidak's m.c. YM155 vs Saline t=3.203, df=70, D2 p=0.0142). Importantly, the inventors observed no changes in locomotor activity between animals immediately after testing mechanical hypersensitivity on DI post-op (FIG. 27).

### Example 5: SARS-COV-2 Induces a Unique, Persistent Transcriptomic Profile in DRGs

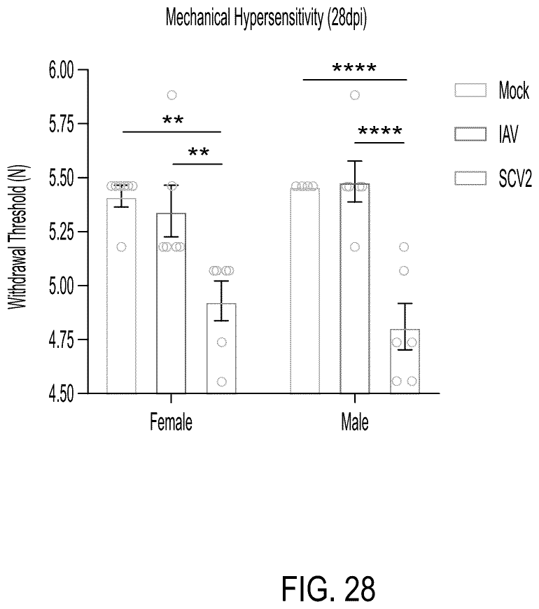

Given that the severity of sensory hypersensitivity during acute infection with SARS COV-2 worsens over time and the existence of persistent sensory symptoms in patients afflicted by long COVID, the inventors set out to determine whether the hamster respiratory model of SARS-COV-2 infection displayed any prolonged sensory phenotypes. To this end, the inventors monitored mechanical hypersensitivity in male and female SARS-COV-2, IAV, and mock treated hamsters at 28 dpi (well-after viral clearance). The inventors findings reveal substantial mechanical hypersensitivity in SARS-COV-2-infected hamsters of both sexes, but normal responses for IAV and mock treated hamsters (FIG. 28: for female groups: one-way ANOVA F(2,15)=8.469, p=0.0035, Tukey's m.c. SARS-COV-2vsMock q=5.385, df=15, p=0.0046: SARS-COV-2vsIAV q=4.605, df=15, p=0.0138: for male groups: one-way ANOVA F(2.15)=22.36, p<0.0001, Tukey's m.c. SARS COV-2vsMock q=8.043, df=15, p=0.0001: SARS-COV-2vsIAV q=8.331, df=15, p<0.0001).

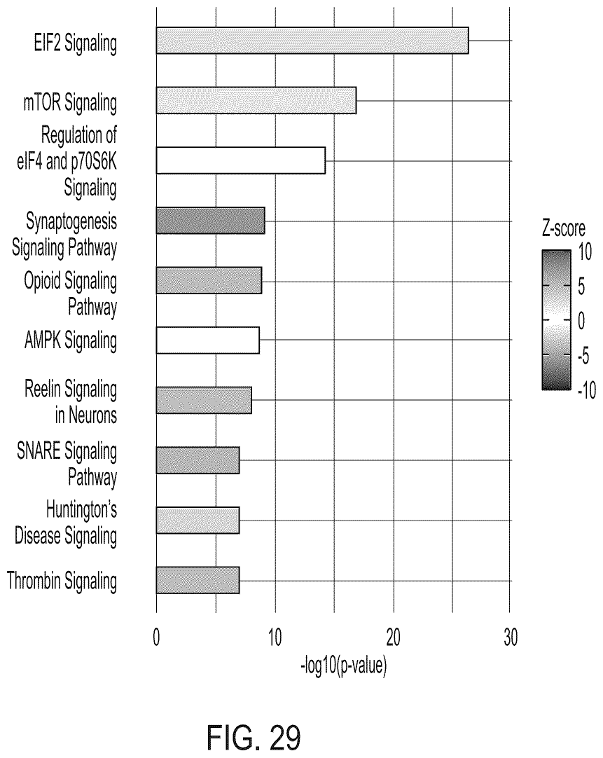

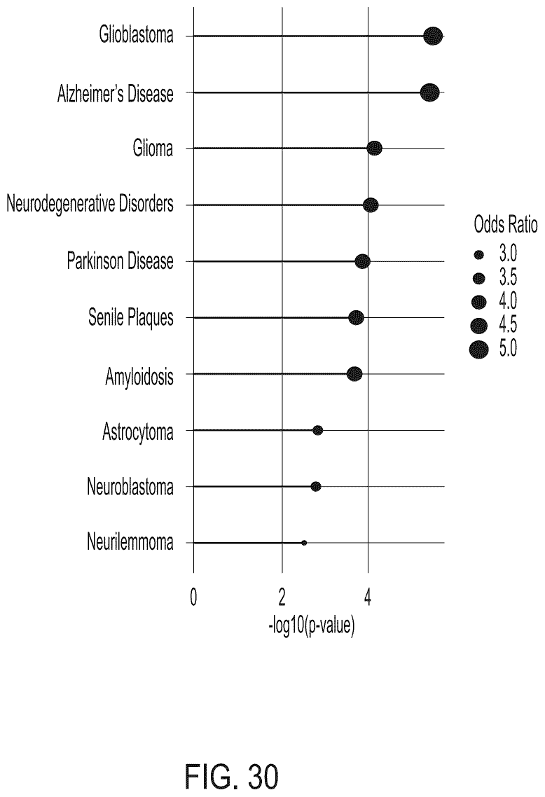

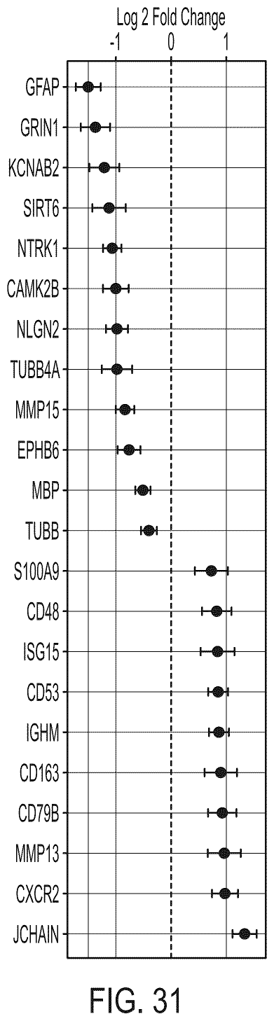

To determine whether longitudinally-altered DRG molecular mechanisms may be responsible for this specific hypersensitivity phenotype, the inventors performed RNA-seq analysis and compared 31 dpi thoracic DRGs between SARS-COV-2 and Mock male animals. Surprisingly, the inventors identified 1065 DEGs (p-adj.<0.1, 170 up, 895 down), which is a much larger number of DEGs than they observed with the 4 dpi SARS-COV-2 DRGs. Ontology analysis of DEGs (nominal p<0.05) also highlighted new and counter-regulated canonical pathways compared to those observed in Idpi and 4 dpi SARS-COV-2 and IAV, including decreased “Synaptogenesis Signaling”, and the involvement of “EIF2 Signaling”, “mTOR Signaling”, “Opioid Signaling”, and “SNARE Signaling” (FIG. 29:-log 10 (p-value)>1.3). Furthermore, use of Enrichr's DisGeNET gateway primarily associated these DEGs with neuro oncological and neurodegenerative conditions, including Glioblastoma, Alzheimer's Disease, Parkinson Disease, and Neurilemmoma (FIG. 30). Key DEGs (p-adj.<0).1) from RNAseq support the inventors' observed maladaptive alterations in canonical neuronal and inflammatory pathways, including changes in gene expression of several tubulin mRNA (Tubb) isoforms, myelin proteins, activity-related channels, extracellular matrix proteins, and cytokine/interferon-related proteins (FIG. 31).

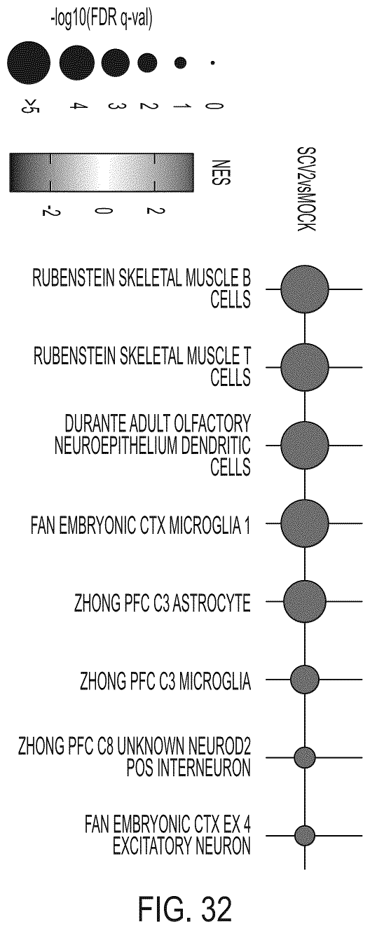

Analysis of predicted cell subtype implications 321 influence on 31 dpi SARS-COV-2 tDRG transcriptomic signatures using GSEA (C8 cell type signature gene set (v7.4)) revealed a positive contribution of pro-inflammatory cells, such as B cells, T cells, and dendritic cells (FIG. 32). Astrocytes, microglia, interneurons, and excitatory neurons contributions were negatively enriched (FIG. 32). Overall, these predictions suggest that 31 dpi SARS-COV-2 tDRGs are undergoing a pro-inflammatory state with inhibited neuronal and glial function. which is reflective of the ontology analysis above.

The inventors sought to determine whether a core group of upstream regulators (URs) may serve as a common target for sensory and perceptive components of pain, as well as affective comorbidities observed in long COVID-19 patients. The inventors performed an IPA UR comparison analysis between the 31 dpi DRG. Striatum, and Thalamus RNA-seq data. the latter two datasets coming from another systemic long-COVID study that the inventors performed in hamsters under the same conditions. The Striatum and Thalamus are all well-cited regions involved in the initiation and maintenance of sensory components of pain, as well as emotional pain signs, such as catastrophizing. Here, the inventors focused on the top common upstream regulators across these regions.

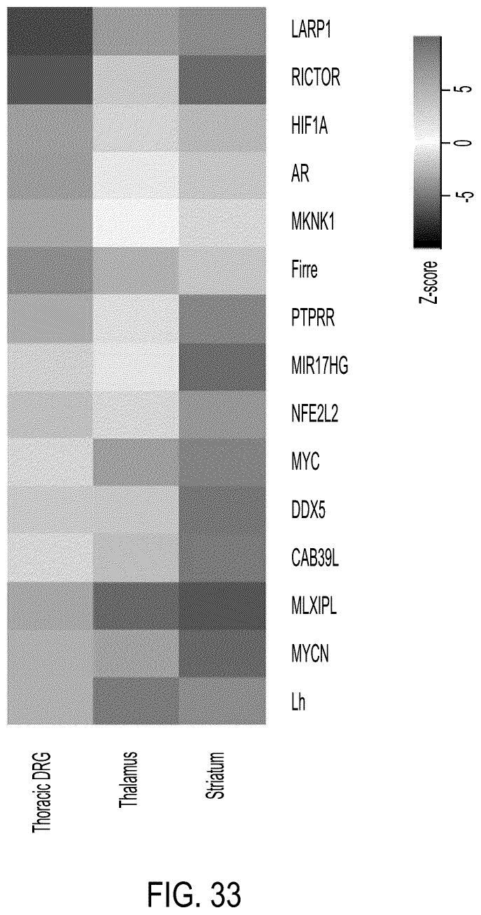

Unexpectedly, a majority of the top 15 URs demonstrated a unidirectional predicted activation/inhibition state between Thalamus and Striatum, but not DRGs (FIG. 33). However, the inventors did observe a common upregulation of PTPRR and miR17hg, as well as a downregulation of FIRRE, between DRG and Thalamus. While PTPRR, a protein tyrosine phosphatase receptor, has not been implicated in pain, human studies have suggested an association between its upregulation and depression. MIR17HG (a long non-coding RNA (lncRNA) involved in cell survival) gene abnormalities have also been reported in Feingold 2 syndrome patients that suffer from chronic myofascial pain and affective symptoms. FIRRE, another lncRNA, has been implicated in spinal cord neuropathic pain mechanisms. Thus, common regulators between the peripheral and central 346 nervous systems may serve as useful targets for both sensory and affective symptoms of long COVID-19.

### Example 6: SARS-COV-2 Infection Causes Transcriptomic Signatures Similar to Persistent Inflammation and Nerve Injury Models in Dorsal Root Ganglia

While their bioinformatic analysis of SARS-COV-2 RNA-seq datasets led to the discovery of targeting ILF3 for treating pain, the inventors also investigated whether a meta-analysis of their data against existing injury datasets would generate a more comprehensive list of pain targets. The inventors therefore compared 1, 4, and 31 dpi thoracic DRG RNA-seq from SARSCoV-2-infected hamsters against gene expression omnibus (GEO) RNA sequencing data from the aforementioned murine SNI and CFA datasets.

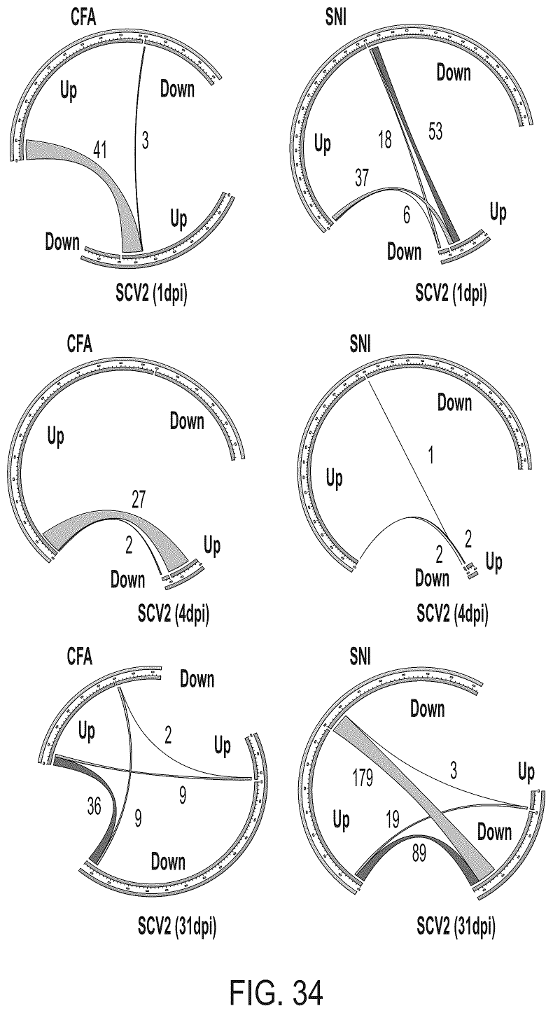

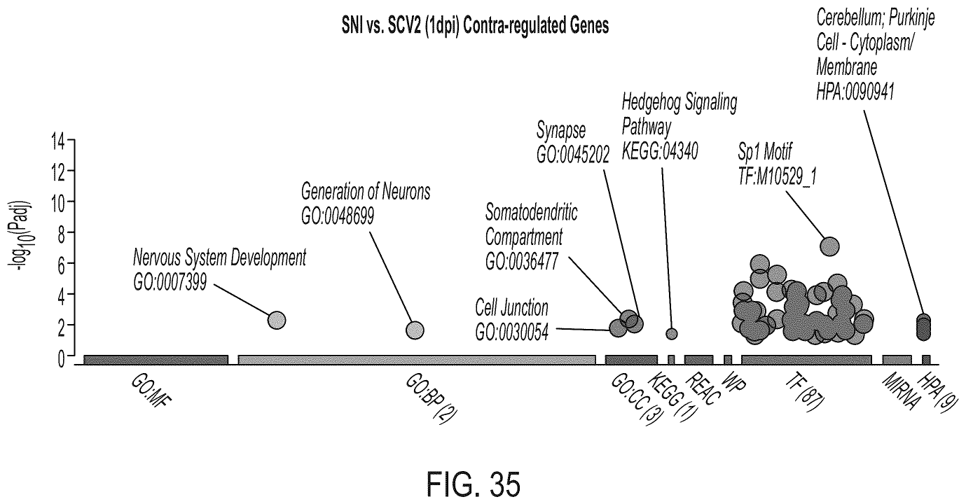

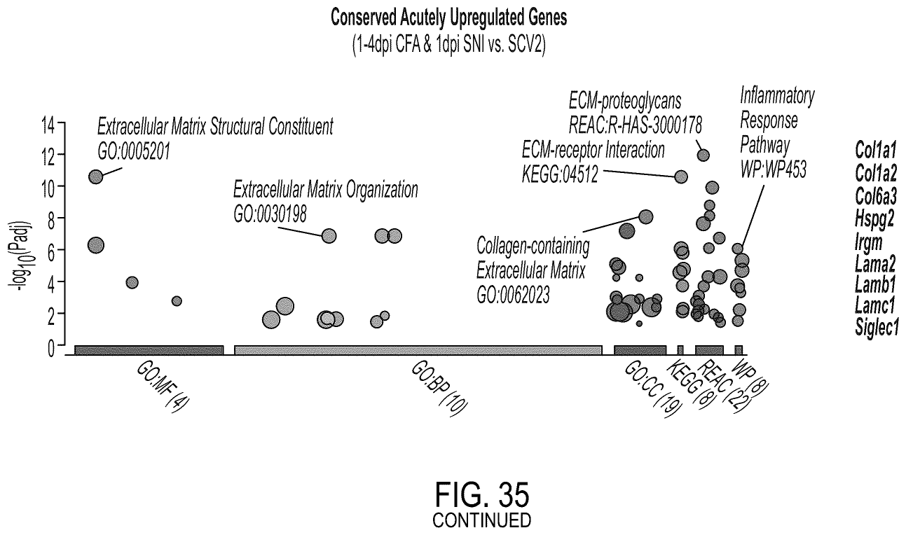

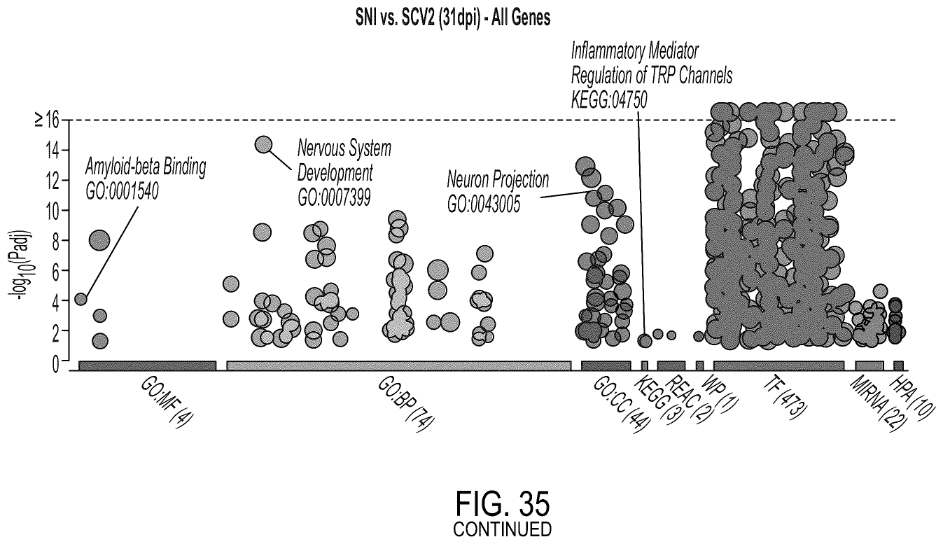

The inventors observed several commonly upregulated genes between SARS-COV-2 and CFA at both 1 and 4 dpi, and only on Idpi when comparing to SNI (FIG. 34). Interestingly, the inventors identified a group of 53 genes that were upregulated by SARS-CoV-2 at Idpi but downregulated by SNI (FIG. 35), g: Profiler associates this gene set with neuroplasticity, particularly in the synaptic/dendritic cellular compartments, and strongly associates the Sp1 transcription factor (implicated in several pro-nociceptive mechanisms) with these genes (FIG. 35). Some of these genes, such as Scn4b, Rhobtb2, Mgll, and Cntfr have been positively associated with sensory hypersensitivity under injury states, suggesting they may be unique mechanisms by which SARS-COV-2 induces mild hypersensitivity. This discovery also highlights potential SNI-induced compensatory anti-nociceptive gene programs. Several genes implicated in neurodevelopment and dendritic plasticity were also upregulated by SARS-COV-2, but they have not yet been studied in pain. Interesting candidates include Olfm1, Fxr2, Atcay, Cplx1, Iqsec1, Dnm1, Clstn1, Rph3a, Scrt1, Ntng2, and Lhfpl4, Ontologies significantly associated with this SARS-COV-2 versus SNI contra-regulated gene list are GO: BP nervous system development (p-adj=0.005994). GO: BP generation of neurons (p372 adj=0.024). GO: CC somatodendritic compartment (p-adj=0.004204). GO: CC synapse (p373 adj=0.01054), and GO: CC cell junction (p-adj=0.02067). The inventors also identified a core set of genes, mostly associated with extracellular matrix remodeling, was commonly upregulated between Idpi SARS-COV-2, CFA, and SNI: Colla1, Colla2, Col6a3, Hspg2. Irgm, Lama2, Lamb1, Lamc1, and Siglec1 (FIG. 35). This agrees with previous literature implicating extracellular matrix remodeling with the maintenance of inflammatory- and nerve injury-associated pain sensation.

Finally, by comparing of all genes regulated by CFA and 31 dpi SARS-COV-2, the inventors discovered a subset of counter-regulated DEGs (36 CFA Up-SARS-COV-2 down: p-adj.<0.1). These genes are implicated in pathways such as myelination/axon ensheathment (Mpz, Mbp, Prx, Fa2h, Dhh, and Mag), semaphorin-regulation of axonogenesis (Sema3g and Sema4g), and extracellular matrix organization (Nid2, Col5a3, Mmp15, Mmp14, Col4al, and Fscn1) (g: profiler GO: BP p adj.<0.05). The inventors also discovered a strong transcriptional counter-regulation between SNI and 31 dpi SARS-COV-2 as well (89 SNI up-SARS-COV-2 down: p-adj.<0.1). This signature was predominantly related with nervous system development, with implicated genes including Mpz, Plec, Prkcg Metrn, Slit1, Brd2, Anksla, Cpne5, Sema4f, Hspg2, Sh3gl1, Prag1, Map6, Mdgal, Fphs, Ppp2r5b, Plod3, Phgdh, Dpysl5, Gpc1, Elavl3, Gpsm1, Marcksl1, Col4a1, Niban2, Carm1, Irs2, Lgi4, Erbb2, Syngap1, and Nlgn2 (g: profiler GO: BP p-adj.<0.05).

The inventors were particularly surprised by the robust overlap of downregulated DEGs between SNI and 31 dpi SARS-COV-2 (179: p-adj.<0.1). Nervous system development and morphogenesis were robust pathway signatures, implicating neuronal plasticity as a key contributor to nerve injury and virus-induced pain states. But the inventors also discovered strongly altered synaptic transmission pathways, with DEGs including Slc7a7, Syngr1, Prkaca, Rab3a, Ntrk1, Nptx1, Stx1b, Jph3, Mapk8ip2, Calm3, Pnkd, Ppp1r9b, Pip5k1c, Cacng7, Dlgap3, Nrxn2, Pink1, Grk2, Ncdn, Cplx2, Camk2b, Grin1, Brsk1, Ache, and Jph4. This gene list reveals that SARS-COV-2 mirrors nerve injury maladaptive mechanisms both through direct modification of neuronal excitability at the membrane level and through modulation of transcriptional regulation elements. These, along with other implicated pathways from the overall SNI-SARS-COV-2 31 dpi comparison, such as amyloid-beta binding and TRP channel modulation.

Combined, this meta-analysis emphasizes SARS-COV-2's ability to recapitulate transcriptional perturbations in the DRG underlying both inflammatory- and nerve injury associated pain states. However, these findings also demonstrate the induction of plasticity associated perturbations that counter those seen in other injury models. These findings support the use of the SARS-COV-2 respiratory infection hamster model as a preclinical chronic pain model, which can be used for the understanding of the evaluation of pharmacological treatments.

### Infection & Local Inflammation Animal Models

One-to two-month-old male golden hamsters (Mesocricetus auratus) were used in all infection experiments, and age-matched female hamsters were included in 31 dpi experiments (Charles River Laboratories, MA). Male hamsters were co-housed on a twelve-hour light-dark cycle and had access to food and water ad libitum. Female hamsters were housed individually to prevent injury due to aggression. Hamster work was performed in a CDC/USDA-approved biosafety level 3 laboratory in accordance with NYU Langone and Icahn School of Medicine at Mount Sinai IACUC protocols. Mice were housed on a twelve-hour light-dark cycle and had access to food and water ad libitum in accordance with the Icahn School of Medicine at Mount Sinai IACUC protocols.

Two-to three-month-old hamsters received an intranasal inoculation of 100 μL of phosphate-buffered saline (PBS) containing 1000 plaque forming units (PFU) of SARS-COV-2, 100,000 PFU of IAV (viral control), or PBS alone (mock control). Hamsters were euthanized by intraperitoneal pentobarbital injection followed by cardiac perfusion with 60 mL PBS.

For studies using models of peripheral inflammation, two-to three-month old mice received 30 μL left hindpaw injections of Complete Freund's Adjuvant (CFA: diluted 1:1 in saline) or IFNB injections (300 U per 25 μL), as described previously. For studies using the post-operative incision model, two-to three-month-old mice received an incision from the posterior plantar surface of the hindpaw to the middle of the paw pads, in which dermis and superficial muscle was cut and dermis was sutured afterwards. CFA and paw incision groups of mice received daily intraperitoneal (i.p.) injections of saline (vehicle) or YM155 (Tocris Biosciences), an Interleukin Enhancer Binding Factor 3 (ILF3) inhibitor (2.5 or 5 mg/kg diluted in saline).

### Von Frey Assay

Hamsters/mice were placed on a raised grid platform in plastic containers and were allowed to habituate to their environment for a minimum of 10 minutes. Afterwards, filaments of ascending forces were applied to the left hindpaw and responses were recorded. A positive response consisted of a hindpaw lift, shake, or lick. Progression to the next filament was determined by recording of positive or negative responses for three out of five applications with each filament. Mechanical withdrawal threshold was defined as the first (for hamsters, to minimize cross-contamination of cohorts by prolonged fomite exposure) or second (mouse, for consistency) filament force at which an animal had three positive responses. All materials utilized for testing of infected hamsters were thoroughly decontaminated between testing of infection groups.

### Hargreaves' Assay

The CFA model induces thermal hypersensitivity for 10-14 days on average. The inventors used the Hargreaves' thermal beam assay to assess the effects of YM155 administration on thermal hypersensitivity associated with left hindpaw CFA injection. Mice were placed on a Hargreaves' platform in plastic containers and were allowed to habituate for 30 minutes. A light beam heat source (IITC Life Science Inc., CA) set to an intensity level of IF=30 was aimed at the left hindpaw for a maximum of 20 seconds (cutoff). Similar to von Frey, paw withdrawal was defined as a hindpaw lift, shake, or lick. Three measurements were recorded and averaged for each hindpaw, with each measurement taking place at least two minutes apart.

### Tissue Processing

Tissues were harvested at 1, 4, and 31 dpi and immediately placed in TRIzol (Invitrogen, MA) for transcriptomic analysis or 4% paraformaldehyde (PFA) in phosphate-buffered saline (PBS) for histology or fluorescent in situ hybridization (RNAscope). Fixed tissues were sucrose converted after 48 hours of 4% PFA fixation in 10% sucrose in PBS (Day 1), 20% sucrose in PBS (Day 2), and 30% sucrose in PBS with 0.01% azide (Day 3). Slide-mounted tissues were paraffin-embedded and sliced to a thickness of 5 microns. Tissue collected for transcriptomic analysis were homogenized in Lysing Matrix A homogenization tubs (MP Biomedicals, CA) for two cycles (40s: 6 m/s) in a FastPrep 24 5 g bead grinder and lysis system (MP Biomedicals, CA). Tissue collected for plaque assays was homogenized in 1 mL PBS in Lysing Matrix A homogenization tubs (MP Biomedicals, CA) for two cycles (40s; 6 m/s).

RNA Isolation & qPCR

RNA was isolated through a phenol: chloroform phase separation protocol as detailed in the TRIzol Reagent User Guide. RNA concentrations were measured by NanoDrop (Thermofisher, MA). 1,000 ng of cDNA was synthesized using the qScript cDNA Synthesis kit (QuantaBio, MA) as detailed in the qScript cDNA Synthesis Kit Manual. Exon-exon-spanning primers targeting as many splice variants as possible were designed with Primer-BLAST (National Center for Biotechnology Information, MD), qPCRs were performed in triplicate with 30 ng of cDNA and a master mix of exon-spanning primers (Supplementary Table 1) and PerfeCTa SYBR Green FastMix ROX (QuantaBio, MA) on an QuantStudio real-time PCR analyzer (Invitrogen, MA), and results were expressed as fold change (2-ΔΔCt) relative to the β-actin gene (Actb).

### Plaque Formation Assay

Plaque assays were performed as described previously (31). Virus was logarithmically diluted in SARS-COV-2 infection medium with a final volume of 200 μL volume per dilution. 12-well plates of Vero E6 cells were incubated for 1 hour at room temperature with gentle agitation every 10 minutes. An overlay comprised of Modified Eagle Medium (GIBCO), 4 mM L-glutamine (GIBCO), 0.2% BSA (MP Biomedicals), 10 mM HEPES (Fisher Scientific), 0.12% NaHCO₃, and 0.7% Oxoid agar (Thermo Scientific) was pipetted into each well. Plates were incubated at 37 degrees C., for 48 hours prior to fixation in 4% PFA in PBS for 24 hours. Plaques were visualized via staining with crystal violet solution (1% crystal violet (w/v) in 20% ethanol (v/v)) for 15 minutes.

### RNAscope In Situ Hybridization

The Fluorescent Multiplex V2 kit (Advanced Cell Diagnostics, CA) was used for RNAscope FISH. Specifically, the inventors used the FFPE protocol as detailed in the RNAscope Multiplex Fluorescent Reagent Kit v2 Assay User Manual. RNAscope probes were as follows: Rbfox3 (NeuN) for pan-neuronal labeling (Mau-Rbfox3-C1) and the Spike gene(S) for SARS COV-2 labeling (V-nCOV2019-S-C3). Opal dyes (Akoya Biosciences, MA) were used for secondary staining as follows: Opal 690 for Cl and Opal 570 for C3. DAPI was used for nuclear staining. Images were taken on an LSM880 confocal microscope (Zeiss, GER) with identical parameters between mock and SARS samples.

### Immunohistochemistry

Immunohistochemistry was performed according to protocols described previously. Briefly, 5 μm sections were cut from FFPE tissues and mounted on charged glass slides. Sections were deparaffinized by immersion in xylene and subsequently submerged in decreasing concentrations of ethanol to rehydrate. Rehydrated sections were submerged in IHC-Tek Epitope Retrieval Solution (Cat #IW-566 1100) and steamed for 45 min in IHC-Tek Epitope Retrieval Steamer (Cat #IW-1102) for antigen retrieval. Tissues were blocked with 10% goat serum and 1% bovine serum albumin in TBS for Ihr at room temperature. Primary antibody (monoclonal murine-derived anti-SARS-COV-2 N protein) was diluted 1:100 in a 1% BSA TBS solution and added to slides. Slides were incubated with primary antibody solution overnight at 4° C. Slides were washed in TBS with 0.025% Triton-X-100 and treated with 0.3% hydrogen peroxide in TBS for 15 min. Slides were washed once again. HRP-conjugated goat anti-mouse IgG secondary antibody (ThermoFisher, Cat #A21426) was diluted 1:5000 and added to slides. Slides incubated with secondary antibody at room temperature for Ihr. Slides were washed twice, and DAB developing reagent (Vector Laboratories, Cat #SK-4105) was added to slides. Slides were dehydrated with increasing concentrations of ethanol and cleared using xylene. Slides were cover slipped, dried, and imaged using brightfield setting on EVOS M5000 inverted microscope.

### RNA Sequencing

RNA was isolated from tissues as previously described above. 500 ng-lug of total RNA per sample was enriched for polyadenylated RNA and prepared for RNA sequencing using the TruSeq Stranded mRNA Library Prep Kit (Illumina) per manufacturer instructions. Samples were sequenced on an Illumina NextSeq 500 platform or by the NYU Langone Genome Technology Center. FASTQ files were then aligned to the golden hamster genome (MesAur 1.0, ensembl) via the RNA-Seq Alignment application (BaseSpace, Illumina). Salmon files were analyzed using DESeq2. For non-ontology analyses, all genes with an adjusted p-value (p-adj) less than 0.1 were considered “Differentially Expressed Genes” (DEGs).

Ontological analysis was performed using g: Profiler and Qiagen Ingenuity Pathway Analysis, targeting genes with a nominal p-value of less than 0.05 to increase analytical power. All visualizations of RNA-seq, differential expression analysis, and ontological analysis data were created by the respective ontological 591 analysis programs or by R using ggplot2, VennDiagram, Circos, pheatmap, ComplexHeatmap, and gplots packages. Gene set enrichment analyses were conducted using the GSEA Java application for Mac (v 4.1.0) (MSigDB: Broad Institute, UC San Diego). Analyses were performed on pre ranked gene lists derived from differential expression data. Genes were ranked by the following statistic:-log 10 (p-value)/sign (log 2FoldChange). GSEA analyses were conducted against the C8 cell type signature gene set (v7.4) provided by the Molecular Signatures Database (MSigDB).

### Meta-Analysis

FASTQ files from Parisien et al. (2019) (99) generated from RNA-seq of DRG tissues from mice subjected to sham (mock), Complete Freund's Adjuvant (CFA), and Spared Nerve Injury (SNI) treatments were obtained from NCBI GEO (GSE111216). Paired end read files were aligned to the Mus musculus transcriptome (GRCm39) and quantified using Salmon (version 1.4.0). Salmon files were analyzed for differentially expressed genes using DESeq2, and all genes expressing a p-adj<0.1 were considered differentially expressed. Differentially expressed genes from murine DRG injury models compared to mock tissues were compared to analogous differentially expressed genes from infected hamster DRG tissues compared to mock hamster DRG tissues. These comparative analyses were visualized using Circos, VennDiagram, and ggplot2. Shared and contra-regulated gene sets highlighted from these analyses were also analyzed for ontology using g: Profiler.

### Statistical Analyses

All statistical analyses outside of sequencing-related assays were performed in GraphPad Prism Version 10. Repeated measure one- and two-way ANOVAs were used to compare the effects of virus type and time of infection on mechanical hypersensitivity, and post-hoc Tukey's multiple comparison test 614 were used to perform timepoint comparisons for the Von Frey assay. Multiple t-tests and two-way ANOVAs were used for qPCR analysis. RNA-seq data was analyzed as described above. Ontology analysis statistics were performed with either Ingenuity Pathway Analysis (IPA), g: Profiler, or Enrichr.

1. A method for preventing pain induction, interrupting pain signaling, or alleviating pain perception in a subject by inhibiting the expression, activity, or function of Interleukin Enhancer Binding Factor 3 (ILF3).

2. The method of claim 1, wherein a type of the pain is selected from the group consisting of: neuropathic pain; postoperative pain; myalgia; chronic pain; emergent pain;
systemic pain; and pain associated with inflammation, infection, post-viral conditions, or disease.

3. The method of claim 1, wherein the preventing, interrupting, or alleviating comprises administering to the subject a polynucleotide, polypeptide, chemical compound, nanoparticles, delayed-release compound, or pharmaceutical.

4. The method of claim 1, wherein the preventing, interrupting, or alleviating comprises administering a compound selected from the group consisting of: YM155, genistein, ivermectin, piroxicam, resveratrol, tamibarotene, thapsigargin, thimerosal, valproic acid, corticosteroids, JAK/TYK inhibitors, anti-IFN antibodies, anti-IFN receptor antibodies, bufexamac, SAR-20347, FLLL32 and vitamin E, or combinations thereof.

5. The method of claim 1, wherein the preventing, interrupting, or alleviating comprises administering a composition comprising YM155.

6. A method for treating pain in a subject by inhibiting the expression or activity of Interleukin Enhancer Binding Factor 3 (ILF3).

7. The method of claim 6, wherein the type of pain is selected from the group consisting of: neuropathic pain; postoperative pain; myalgia; chronic pain; emergent pain; systemic pain; and pain associated with inflammation, infection, post-viral conditions, or disease.

8. The method of claim 6, wherein the treating comprises administering to the subject a polynucleotide, polypeptide, chemical compound, nanoparticles, delayed-release compound, or pharmaceutical.

9. The method of claim 6, wherein said inhibiting is achieved by administering a compound selected from the group consisting of: YM155, genistein, ivermectin, piroxicam, resveratrol, tamibarotene, thapsigargin, thimerosal, valproic acid, corticosteroids, JAK/TYK inhibitors, anti-IFN antibodies, anti-IFN receptor antibodies, bufexamac, SAR-20347, FLLL32 and vitamin E, or combinations thereof.

10. A method for treating localized pain in a subject by inhibiting the expression or activity of Interleukin Enhancer Binding Factor 3 (ILF3).

11. The method of claim 10, wherein the type of pain is selected from the group consisting of: neuropathic pain; postoperative pain; myalgia; chronic pain; emergent pain;
systemic pain; and pain associated with inflammation, infection, post-viral conditions, or disease.

12. The method of claim 10, wherein the treating comprises administering to the subject a polynucleotide, polypeptide, chemical compound, or pharmaceutical.

13. The method of claim 10, wherein the inhibiting comprises administering a compound selected from the group consisting of: YM155, genistein, ivermectin, piroxicam, resveratrol, tamibarotene, thapsigargin, thimerosal, valproic acid, corticosteroids, JAK/TYK inhibitors, anti-IFN antibodies, anti-IFN receptor antibodies, bufexamac, SAR-20347, FLLL32 and vitamin E, or combinations thereof.

14. The method of claim 6, wherein the treating comprises administering a composition comprising YM155.

15. The method of claim 6 wherein said compound is chemically modified to stabilize it during or after administration.

16. The method of claim 6 wherein said compound is administered via encapsulation in, containment in, or protection with nanoparticles, vesicles, polymers, phospholipids, or combinations thereof.

17. The method of claim 6 wherein said compound is a delayed release formulation.

18. The method of claim 10 wherein the treating comprises administering a composition comprising YM155

19. The method of claim 10 wherein said compound is chemically modified to stabilize it during or after administration.

20. The method of claim 10 wherein said compound is administered via encapsulation in, containment in, or protection with nanoparticles, vesicles, polymers, phospholipids, or combinations thereof.
